(19) United States
(12) Patent Application Publication
(10) Pub. No.: US 2025/0277785 A1
(43) Pub. Date: Sep. 4, 2025

(54) MICROFLUIDIC DEVICES FOR EVALUATING FIBROSIS IN MATERIAL IMPLANTATION AND CANCER

(71) Applicant: THE UNITED STATES OF AMERICA AS REPRESENTED BY THE SECRETARY. DEPARTMENT OF HEALTH AND HUMAN SERVICE, Bethesda, MD (US)

(72) Inventors: Kaitlyn Noelle SADTLER, Bethesda, MD (US); Parinaz FATHI, Bethesda, MD (US); Nicole MORGAN, Bethesda, MD (US); Paniz REZVAN SANGSARI, Bethesda, MD (US)

(21) Appl. No.: 18/859,345

(22) PCT Filed: Apr. 26, 2023
(86) PCT No.: PCT/US2023/020026

§ 371 Date: Oct. 23, 2024

Related U.S. Application Data
(60) Provisional application No. 63/335,527, filed on Apr. 27, 2022.

(51) Int. Cl. G01N 33/50; B01L 3/00

(52) U.S. Cl. CPC G01N 33/5044; B01L 3/502715; G01N 33/5091; B01L 2300/0663; B01L 2300/087; B01L 2300/16; B01L 2300/18

(57) ABSTRACT

Fibrotic diagnostic systems, subsystems, and components thereof are provided. A fibrotic diagnostic system can comprise a microfluidic device. The fibrotic diagnostic system can be preloaded with one or more components. For example, the fibrotic diagnostic system can comprise the one or more reagents and a target object, for example a tissue, or a medical device, or both intended for implantation. The fibrotic diagnostic system can comprise one or more additional components and subsystems, for example, a detector comprising a sensor configured to detect fibrosis of the target object, a thermal subsystem, a fluidic subsystem, a processor, or a user interface, or any combination thereof. Kits comprising one or more components of a fibrotic diagnostic system are provided. Screening methods for identifying therapeutics are provided that utilize a fibrotic diagnostic system or component thereof are provided. Non-transitory computer-readable media storing a program are further provided.

### CROSS-REFERENCE TO RELATED APPLICATIONS

This is an International Application under the Patent Cooperation Treaty, claiming priority to U.S. Provisional Patent Application No. 63/335,527, filed 27 Apr. 2022, the contents of which are incorporated herein by reference in their entirety

### BACKGROUND

### Field

The present disclosure relates to systems, devices, and methods for analyzing fibrosis associated with foreign bodies and therapeutic approaches based on that analysis.

### Description of Related Art

The foreign body response leads to fibrosis of implants and cancer tumors, which can cause multiple negative effects such as hypertrophic scarring, excessive inflammation, and a reduction in the efficacy of cancer therapeutics. During the fibrosis formation process, crosstalk primarily between macrophages and fibroblasts leads to isolation of biomaterials from the native tissue. Traditionally, these interactions have been evaluated by in vivo placement of foreign objects in mouse or rat models. Available in vitro models attempt to mimic scarring including associated fibrosis. These models can provide relevant data but fail to provide a sufficient model of the interactions that take place in the human body, and consequently can have limited predictive value. Solutions to these technical problems are provided by the present disclosure.

### BRIEF SUMMARY

Fibrotic diagnostic systems, subsystems, and components thereof are provided by the present disclosure. The fibrotic diagnostic system can comprise a microfluidic device. The microfluidic device can comprise a device housing comprising a device interior, a plurality of channels within the device interior, a plurality of septa separating the channels from one another, and a plurality of a channel ports in the device housing that are in fluid communication with the plurality of channels. The plurality of channels can comprise, for example, first, second, and third channels. The first channel can be a central channel and the second and third channels can be lateral channels on opposing sides of the central channel. The first channel can be configured to receive a target object, a matrix, and a first biological composition configured to promote fibrosis of the target object. The second channel can be configured to receive a second biological composition configured to promote fibrosis of the target object. The first septum can separate the first and second channels. The third channel can be configured to receive a third biological composition configured to promote fibrosis of the target object. The second septum can separate the first and third channels. The plurality of channel ports can comprise any number of channel ports with a minimum of two channel ports. The plurality of channel ports can comprise one or more sets of channel ports. The fibrotic diagnostic system can be configured to form a gradient of at least one type of molecule across the first channel, the second channel, or the third channel, or any combination thereof. The fibrotic diagnostic system can be preloaded with one or more components. For example, the fibrotic diagnostic system can comprise the matrix, the first biological composition, the second biological composition, or the third biological composition, or any combination thereof. The fibrotic diagnostic system can comprise the target object. The fibrotic diagnostic system can comprise one or more additional components and subsystems, for example, a detector comprising a sensor configured to detect fibrosis of the target object, a thermal subsystem, a fluidic subsystem, a processor, or a user interface, or any combination thereof. Kits comprising one or more components of a fibrotic diagnostic system are provided. Screening methods employing a fibrotic diagnostic system or component thereof are provided. Screening methods comprise, for example, methods of screening for anti-fibrotic agents or anti-cancer agents. Non-transitory computer-readable media storing a program comprising instructions to perform a method of the present disclosure, or for controlling a system of the present disclosure are further provided.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1A is a perspective view of a microfluidic device in accordance with the present disclosure.

FIG. 1B is a plan view of the microfluidic device shown in FIG. 1A.

FIG. 2A is a perspective view of a fibrotic diagnostic system showing a microfluidic device disengaged from a detector in accordance with the present disclosure.

FIG. 2B is a perspective view of the fibrotic diagnostic system of FIG. 2A showing a microfluidic device engaged with a detector in accordance with the present disclosure.

FIG. 3 is a plan view of a microfluidic device in accordance with the present disclosure.

FIG. 4 is a plan view of a microfluidic device in accordance with the present disclosure.

FIG. 5 is a plan view of a microfluidic device in accordance with the present disclosure.

FIG. 6A is a top perspective view of a microfluidic device in accordance with the present disclosure.

FIG. 6B is a front perspective view of the microfluidic device shown in FIG. 6A.

FIG. 6C is an enlarged perspective view of a portion of the microfluidic device shown in FIG. 6A.

FIG. 6D is an enlarged plan view of a portion of the microfluidic device shown in FIG. 6A.

FIG. 6E is a further enlarged plan view of a portion of the enlarged view shown in FIG. 6D.

FIG. 7A is a schematic cross-sectional view of a loaded microfluidic device at a first time point in accordance with the present disclosure.

FIG. 7B is a schematic cross-sectional view of a loaded microfluidic device shown in FIG. 7A at a second time point.

FIG. 8 is a schematic diagram of a fibrotic diagnostic system in accordance with the present disclosure.

FIG. 9 is a schematic diagram of a fibrotic diagnostic system in accordance with the present disclosure.

FIG. 10 is a schematic diagram of a fibrotic diagnostic system in accordance with the present disclosure.

FIG. 11, parts A, B, and C show magnified images of cells stained with picrosirius red in accordance with the present disclosure.

FIG. 12 shows magnified fluorescence images of cells grown in 96-well plates, uncoated or coated with fibronectin, with L-ascorbic acid, with or without TGF-β1, and fixed with 4% paraformaldehyde (PFA) in accordance with the present disclosure.

FIG. 13 shows magnified fluorescence images of cells grown in 96-well plates, uncoated or coated with fibronectin, with L-ascorbic acid, with or without TGF-β1, and fixed with methanol in accordance with the present disclosure.

FIG. 14 depicts graphs of quantified fluorescence intensity at 4× and 10× magnification for collagen I and α-SMA channels of cells grown in accordance with the present disclosure.

FIG. 15 depicts graphs of quantified fluorescence intensity at 20× and 40× magnification for collagen I and α-SMA channels of cells grown in accordance with the present disclosure.

FIG. 16 shows magnified fluorescence images of cells grown in eight chamber glass slides, uncoated or coated with fibronectin, with L-ascorbic acid, with or without TGF-β1, and fixed with 4% paraformaldehyde (PFA) in accordance with the present disclosure.

FIG. 17 shows magnified fluorescence images of cells grown in eight chamber glass slides, uncoated or coated with fibronectin, with L-ascorbic acid, with or without TGF-β1, and fixed with methanol in accordance with the present disclosure.

### DETAILED DESCRIPTION

A fibrotic diagnostic system is provided by the present disclosure. The fibrotic diagnostic system can comprise a microfluidic device. The microfluidic device can comprise a device housing comprising a device interior, a plurality of channels within the device interior, a plurality of septa separating the channels from one another, and a plurality of a channel ports in the device housing in fluid communication with the plurality of channels. The plurality of channels can comprise any number of channels with a minimum of two channels. The plurality of channels can be, for example, 2, 3, 4, 5, 6, 7, 8, 9, or 10 channels, or more than 10 channels. The number of septa can be, for example, one fewer than the number of channels. For example, the plurality of channels can comprise first, second, and third channels with first and second septa. The number of septa may be, for example, 1, 2, 3, 4, 5, 6, 7, 8, or 9 septa, or more than 9 septa. The first channel can be a central channel and the second and third channels can be lateral channels on opposing sides of the central channel (the first channel). The first channel can be configured to receive a target object, a matrix, and a first biological composition configured to promote fibrosis of the target object. The second channel can be configured to receive a second biological composition configured to promote fibrosis of the target object. The first septum can separate the first and second channels. The first septum can comprise a first plurality of openings providing fluid communication between the first and second channels. The third channel can be configured to receive a third biological composition configured to promote fibrosis of the target object. The second septum can separate the first and third channels. The second septum can comprise a second plurality of openings providing fluid communication between the first and third channels. The plurality of channel ports in the device housing in fluid communication with the plurality of channels can comprise any number of channel ports with a minimum of two channel ports. The plurality of channel ports can comprise one or more sets of channel ports with a set belonging to a particular channel. For example, the plurality of channel ports can comprise a first set of channel ports in fluid communication with the first channel, a second set of channel ports in fluid communication with the second channel, and a third set of channel ports in fluid communication the third channel, wherein each set of channel ports comprises one or more channel ports.

The fibrotic diagnostic system can be configured to form a gradient of at least one type of molecule across the first channel, the second channel, or the third channel, or any combination thereof. For example, the at least one type of molecule can comprise a chemotactic molecule. The chemotactic molecule can promote the movement of at least one cell type present in the first biological composition, the second biological composition, or the third biological composition, or any combination thereof. The at least one type of molecule can comprise a growth factor. The growth factor can promote the growth of a cell population and in so doing form a gradient comprising that cell population. The first biological composition, the second biological composition, or the third biological composition, or any combination thereof can comprise the at least one type of molecule.

The plurality of channel ports can comprise any number of channel ports with a minimum of two channel ports. Channels can share one or more channel ports. Channels can have one or more exclusive channel ports. The plurality of channel ports can comprise at least one channel port for each channel. The plurality of channel ports can comprise at least two channel ports for at least one channel. The plurality of channel ports can comprise one or more sets of channel ports with a set of channel ports belonging to a particular channel. For example, the second and third sets of channel ports each comprise two or more channel ports. Each set of channel ports can comprise any desired number of channel ports, for example, at least one channel port, two or more channel ports, three or more channel ports, or four or more channel ports. The second and third sets of channel ports each comprise a first channel port and a second channel port, for example, the first channel port proximal to a first end of its respective channel and the second channel proximal to a second end of its respective channel. Each of the sets of channel ports can comprise a first channel port and a second channel port, for example, the first channel port proximal to a first end of its respective channel and the second channel proximal to a second end of its respective channel. The first channel can comprise a central channel port configured to receive the target object into the first channel. The central channel port can be between a first end of the first channel and a second end of the second channel. For example, the central channel port can be equidistant between the first and second ends (termini of first channel). One or more of the plurality of channel ports can comprise a seal, valve, or closure, or any combination thereof configured for movement between an open state and a closed state. One or more of the plurality of channel ports can be configures to connect to a fluidic subsystem for transfer of one or more components into the microfluidic device, or out of the microfluidic device, or both. A channel port can be configured as an inlet, or an outlet, or both.

One or more retention elements can be located in one or more channel of the multifluidic device. For example, one or more retention elements can be located in one or more of the first, second, and third channels. The retention element can be configured to retain at least one component insertable into a channel of the microfluidic device, for example, one or more of the first, second, and third channels. The retention element can be of any desired design or combination of designs. A retention element can be permanent or removable. A retention element can be moveable within the microfluidic device or moveable into or out of the microfluidic device. The retention element can be retractable. The retention element can comprise, for example, a modification of a channel surface. The modification can comprise, for example, a biomolecule. The biomolecule can comprise a polypeptide, a polysaccharide, a lipid, or a polynucleotide, or any combination thereof. The modification can comprise, for example, a polymer, a metal, or a ceramic, or any combination thereof. The device housing can comprise a polymer, a metal, or a ceramic, or any combination thereof. For example, the device housing can comprise a first polymer and the modification can comprise a second polymer. The first and second polymers can differ from each other in chemical composition. The modification can comprise, for example, a physical structure. The physical structure and the device housing can comprise a common material. The modification can comprise a container for receiving the target object. The container can comprise, for example, a recess in the channel surface. The container, indentation, or other physical structure can have any desired geometry, or size, or both. For example, the container, indentation, or physical structure can be circular, elliptical, triangular, square, rectangular, pentagonal, hexagonal, heptagonal, octagonal, or trapezoidal, or any combination thereof. The width, diameter, height, or depth, or any combination thereof of the container, indentation, or other physical structure can be less than about 1.0 μm, from about 1.0 μm to about 1.0 cm, from about 10 μm to about 100 mm, from about 50 μm to about 50 mm, from about 50 μm to about 5.0 mm, from about 50 μm to about 9.5 mm, from about 100 μm to about 1.0 mm, or greater than 1.0 cm. The physical structure can comprise a plurality of physical structures. For example, the plurality of physical structures can comprise a plurality of recesses in the channel surface, or a plurality of protrusions from the channel surface, or both.

The microfluidic device and any component thereof can have any desired geometry, shape, size, dimensions, or relative scale, or any combination thereof. For example, the length, or the width, or both microfluidic device can be less than 1.0 mm, from about 1.0 mm to about 10 cm, from about 2.5 mm to about 5.0 cm, from about 5.0 mm to about 2.5 cm, from about 7.5 mm to about 1.0 cm, or greater than 10 cm. The thickness of the microfluidic device can be less than about 0.001 mm, from about 0.001 mm to about 1.0 cm, from about 0.01 mm to about 0.5 cm, from about 0.1 mm to about 0.1 cm, from about 1.0 mm to about 0.5 cm, from about 10 mm to about 100 mm, or greater than 1.0 cm. The first channel, the second channel, or the third channel, or any combination thereof can comprise a curvilinear cross-section, or a rectilinear cross-section, or both. A curvilinear cross-section can be circular or elliptic. The first channel, the second channel, or the third channel, or any combination thereof can comprise a constant width, or a constant diameter, or both. The first channel, the second channel, or the third channel, or any combination thereof can comprise a variable width, or a constant diameter, or both. A width, a diameter, or a height, or any combination thereof of a channel can be less than about 1.0 μm, from about 1.0 μm to about 1.0 cm, from about 10 μm to about 0.5 cm, from about 50 μm to about 1.0 cm, from about 100 μm to about 0.1 cm, from about 1.0 mm to about 500 mm, from about 10 mm to about 100 mm, or greater than 1.0 cm, or any value therebetween, or any intervening range. Two or more channels of the plurality of channels can have a common length. A channel length, or width, or both can be less than 1.0 mm, from about 1.0 mm to about 10 cm, from about 2.5 mm to about 5.0 cm, from about 5.0 mm to about 2.5 cm, from about 7.5 mm to about 1.0 cm, or greater than 10 cm, or any value therebetween, or any intervening range. A width of a channel, or a device, or both can be, for example, about 5.0 mm, about 6.0 mm, about 7.0 mm, about 8.0 mm, about 9.0 mm, or about 10 mm, or any value therebetween, or any intervening range. Two or more channels of the plurality of channels are parallel to one another. The first channel can be between the second and third channels. Two or more of the first, second, and third channels can differ from another with respect to a volume, a surface area, a cross-sectional area, length, a width, or a diameter, or any combination thereof. For example, a width, a diameter, or both of the first channel can be greater than a width, a diameter, or both of the second channel, or of the third channel, or of both. A cross-sectional area, a width, a diameter, or any combination thereof of one or more of the first, second, and third channels can differ along its length.

Any channel can comprise any number of segments. Any two segments can be continuous or non-continuous. Any two segments can be adjacent or non-adjacent. Any two adjacent segments can be open, closed, partially closed, or of variable connection, or any combination thereof. For example, two adjacent segments, or two adjacent channels, or both can be separated by a valve. Any type of valve can be employed. A valve can be a one-way valve or a two-way valve. A channel can be divided, for example, into three segments. For example, one or more of the first, second, and third channels can comprise a first peripheral segment proximate a first end of the channel, a second peripheral segment proximate a second end of the channel, and a central segment between the first and second peripheral segments. A channel can have a central longitudinal axis, and each segment can have a respective central longitudinal axis. These axes can serve as references for the relationship, for example, orientation between respective segments of a given channel as well as between segment of different, for example, adjacent, channels. The central peripheral segment can comprise a cross-sectional area, a width, or a diameter, or any combination thereof that is greater than a cross-sectional area, a width, or a diameter, or any combination thereof of one or both of the first and second peripheral segments. The central peripheral segment of the first channel can comprise a cross-sectional area, a width, or a diameter, or any combination thereof that is greater than a cross-sectional area, a width, or a diameter, or any combination thereof of one or both of the first and second peripheral segments of the first channel. One or both of the first and second peripheral segments of a channel can be colinear with the central segment in relation to their respective central longitudinal axes. One or both of the first and second peripheral segments can form an angle other than 180° with the central segment in relation to their respective central longitudinal axes. For example, the angle can comprise a right angle, an acute angle, or an obtuse angle, or any combination thereof. Two or more channels of the plurality of channels can lie in a common plane, for example, the first, second, and third channels of the plurality of channels can lie in a common plane.

The microfluidic device can comprise any desired number of septa. The number of septa can be, for example, one fewer than the number of channels. For example, the plurality of channels can comprise first, second, and third channels with first and second septa. Any two septa can vary or be the same with respect to a particular parameter, for example, a geometry, a material, or design, or any combination thereof. For example, a length of the first septum can be less than a length of the first channel, or a length of the second channel, or both. A length of the second septum can be less than a length of the first channel, or a length of the third channel, or both. A septum can comprise a membrane. For example, the first septum, the second septum, or both can comprise a membrane. The membrane can comprise a porous membrane. A pore width, or diameter, or both, can be less than about 1.0 nm, from about 1.0 nm to about 1.0 cm, from about 10 nm to about 1.0 mm, from about 10 nm to about 500 μm, from about 50 nm to about 100 μm, from about 100 nm to about 10 μm, or greater than 1.0 cm, or any value therebetween, or any intervening range. The porous membrane can comprise, for example, polydimethylsiloxane (PDMS), polytetrafluoroethylene (PTFE), polyethylene terephthalate (PET), or the like. The membrane can comprise a semipermeable membrane. The membrane can comprise a biological membrane, or a synthetic membrane, or both.

The first septum can comprise a first plurality of columns separated by the first plurality of openings, the second septum can comprise a second plurality of columns (pillar, micropillars, or the like) separated by the second plurality of second openings, or both. Any desired number, size, shape, and spacing of columns can be used. For example, the first plurality of columns, or the second plurality of columns, or both can have a rectilinear cross-section, a curvilinear cross-section, or both. For example, the first plurality of columns, or the second plurality of columns, or both can be circular, elliptical, polygonal, square, triangular, rectangular, pentagonal, hexagonal, heptagonal, octagonal, or trapezoidal, or any combination thereof with respect to general shape, or cross-sectional shape, or both. A width, diameter, a height, or a depth, or any combination thereof of a column can be less than 1.0 μm, from about 1.0 μm to about 1.0 mm, from about 5.0 μm to about 300 μm, from about 5.0 μm to about 500 μm, from about 5.0 to about 700 μm, from about 10 μm to about 250 μm, from about 25 μm to about 100 μm, or greater than 1.0 mm, or any value there between, or any intervening range. The columns can be extension of any surface or combination of surfaces of a channel or adjacent channels. Columns can extend from a lateral channel wall, a channel ceiling, or a channel floor, or any combination thereof. A column can partially or completely span a channel width, or a channel diameter, or both. Number, placement, size, and shape of columns can affect flow patterns such as eddies, laminar flow, and turbulent flow.

Columns can be evenly or irregularly spaced to form the plurality of openings therebetween. An average, for example, a mean distance, between adjacent columns can be less than 1.0 μm, from about 1.0 μm to about 1.0 mm, from about 5.0 μm to about 300 μm, from about 5.0 to about 500 μm, from about 5.0 μm to about 700 μm, from about 10 μm to about 250 μm, from about 25 μm to about 100 μm, or greater than 1.0 mm, or any value there between, or any intervening range. The first plurality of openings can differ in number, size, shape, or spacing, or any combination thereof; or the openings of the second plurality of openings can differ in number, size, shape, or spacing, or any combination thereof; or both. The openings of the first plurality of openings can differ in number, size, shape, or spacing, or any combination thereof along one or more dimension of the first septum; or the openings of the second plurality of openings can differ in number, size, shape, or spacing, or any combination thereof along one or more dimension of the second septum; or both. The first plurality of openings and the second plurality of openings can differ from one another.

The device housing can comprise a substrate and the plurality of channels and the plurality of channel ports are formed in the substrate. The device housing can comprise a polymer, a metal, or a ceramic, or any combination thereof. The polymer can be, for example, a silicone. The silicone can comprise polydimethylsiloxane (PDMS) or the like, for example, a material described in Campbell et al., “Beyond Polydimethylsiloxane: Alternative Materials for Fabrication of Organ-on-a-Chip Devices and Microphysiological Systems,” ACS Biomater. Sci. Eng., 7, 7, 2880-2899 (2021), which is incorporated by reference herein in its entirety. The device housing can be fabricated from polydimethylsiloxane (PDMS), polymethylmethacrylate (PMMA), agarose, alginate, hyaluronic acid, gelatin, other polymeric materials, a glass, or quartz crystal, or other optically transparent material, or any combination thereof. The device housing can comprise a flexible material, or a rigid material, or both. The device housing can comprise a conductor, or an insulator, or both.

Any surface of the microfluidic device or contents thereof can be coated with one or more materials. The interior, exterior, or ports, or any combination thereof of the microfluidic device can be coated. The substrate from which the microfluidic device can comprise a first material and one or more channel surfaces can comprise a second material differing from the first material. The second material can be distinct from the contents of one or more channels in which it is present. The second material can be a coating on the first material. One or more channel surfaces can be coated with a plurality of different materials. One or more channel surfaces can be coated with a plurality of coatings. At least two of the plurality of coatings can differ in material composition. The substrate can comprise a first material, and the plurality of coatings can comprise a first coating comprising a second material adjacent the substrate on one or more channel surfaces and a second coating comprising a third material overlaying the first coating. The first and second materials can differ from one another and the second and third materials differ from one another. The first, second, and third materials differ from one another. The plurality of coatings can comprise at least two layers differing in average thickness from one another. A coating can have one, two, three, four, five, or ten, or more layers. A thickness, or patterning, or a combination thereof of a coating on one or more channel surfaces can differ along the lengths of one or more channels. Two or more of the channels can differ in material composition of their surface coatings. The first channel can comprise a first coating, the second channel can comprise a second coating, and the third channel can comprise a third coating-the first, second, and third coating differing in a material composition, a thickness, or patterning, or a combination thereof. The thickness of a coating or layer can be one atom, one molecule, less than 1.0 nm, from about 1.0 nm to about 1.0 mm, from about 10 nm to about 100 nm, from about 50 nm to about 5.0 μm, from about 0.5 μm to about 50 μm, from about 1.0 μm to about 10 μm, from about 10 μm to about 100 μm, from about 0.5 mm to about 1.0 mm, or greater than 1.0 mm, or any intervening thickness, or any range therebetween. The patterning can be linear, rectilinear, curvilinear, diagonal, axial, radial, spiral, cross-hatched, spotted, diffuse, random, lateral interior of a channel, a bottom interior of a channel, a bottom interior half of a channel, a top interior of a channel, a top interior half of a channel,

The first, second, and third channels can be coated with the same material. The one or more coatings can comprise an organic material, or an inorganic material, or both. The one or more coatings can comprise a polymer. The one or more coatings can comprise a biomolecule. The one or more coatings can comprise a protein, or a nucleic acid, a lipid, or a carbohydrate, or any combination thereof. The one or more coatings comprise a fibronectin, an extracellular matrix-derived composition, a basement membrane extract, a decellularized extracellular matrix, a solubilized basement membrane preparation, a mixture of extracellular matrix proteins, a tumor derived composition, Matrigel, or a hyaluronic acid, or any combination thereof. The substrate can comprise silicon and one or more channel surfaces can be coated with a coating comprising a silane. The one or more channel surfaces can be subjected to silanization. The one or more channel surfaces can be treated with plasma. The plasma can comprise oxygen. The one or more coatings can be applied by emersion, spraying, brushing, injection, precipitation, polymerization, or vapor deposition, or any combination thereof. The one or more channel surfaces can comprise a laminate. The one or more channel surfaces can comprise an adhesive.

One or more surfaces can be etched, peened, abraded, or smoothed, or a combination thereof, or otherwise modified, to form patterns, for example, those described herein for coatings, ridges, grooves, bumps, or holes, or other surface feature, or any combination thereof. Physical surface features can have dimensions as described herein for coatings. One or more coatings can be combined one or more physical surface features. Coatings and physical surface features can be hydrophilic, or hydrophobic, or both. Coatings and physical surface features can affect flow within one or more channels of the microfluidic device. Coatings and physical surface features can be applied to other surfaces of the microfluidic device in addition to or in the alternative to channel surfaces.

The device housing can comprise a transparent material, a translucent material, a tinted material, a colored material, a polarizing material, a material that selectively transmits wavelengths of electromagnetic radiation, a material that selectively refracts wavelengths of electromagnetic radiation, a material that selectively reflects wavelengths of electromagnetic radiation, or an opaque material, or any combination thereof. The device housing can comprise a backing. The backing can comprise, for example, any material that enables the transmission of light for fluorescence and optical microscopy. The device housing can comprise a window permitting a view of the device interior. The view can be of one or more channel of the plurality of channels, or a component thereof, or content thereof, or any combination thereof. For example, the view can comprise a view of the first channel, or the target object, or both. The device housing can comprise a plurality of windows permitting a plurality of views of the device interior. The microfluidic device can be fabricated using any technique or combination of techniques. For example, the microfluidic device can be fabricated using molding, three-dimensional printing, or etching, or any combination thereof. Three-dimensional printing can include, for example, selective laser sintering (SLS), fused deposition modeling (FDM), or photopolymerization, or any combination thereof.

The target object can comprise an organic material, or an inorganic material, or both. The target object can comprise a cell, a tissue, or a medical device, or any combination thereof. The cell or tissue can be a human cell. The cell or tissue can be mammalian other than human, for example, rat, murine, hamster, monkey, ape, canine, feline, ovine, bovine, equine, or porcine. The target object can comprise a spheroid, an organoid, an alginate-based biomaterial, a scaffold-supported cellular structure, or a bio-printed structure, or any combination thereof. A spheroid can be produced using any suitable method, for example, hanging drop, hydrogels, rotary cell cultures, cell suspensions with nanofibers, magnetic levitation, microfluidics, or bioprinting, or any combination thereof. The target object can comprise a single-cell suspension, a suspension of spheroids, or a suspension of organoids, or any combination thereof.

The target object can comprise a neoplastic cell, a malignant cell, or a benign cell, or any combination thereof. The target object can comprise a cancer cell, or a cancerous tissue, or both. For example, the cancer can be a pre-malignant growth, malignant growth, or tumor caused by abnormal and uncontrolled cell division that can be metastatic or non-metastatic. The cancer can be, for example, a breast cancer, a prostate cancer, a lung cancer, a colon cancer, a rectal cancer, a urinary bladder cancer, a non-Hodgkin a lymphoma, a melanoma, a renal cancer, a pancreatic cancer, a cancer of the oral cavity, a pharynx cancer, an ovarian cancer, a thyroid cancer, a stomach cancer, a brain cancer, a glioma, a multiple myeloma, an esophageal cancer, a liver cancer, a cervical cancer, a larynx cancer, a cancer of the intrahepatic bile duct, an acute myeloid leukemia, a soft tissue cancer, a small intestine cancer, a testicular cancer, a chronic lymphocytic leukemia, a Hodgkin lymphoma, a chronic myeloid cancer, an acute lymphocytic cancer, a cancer of the anus, a cancer of the vulva, a cancer of the neck, a cancer of the gallbladder, a malignant mesothelioma, a bone cancer, a cancer of the joints, a hypopharynx cancer, a cancer of the eye, a cancer of the nose, a cancer of the nasal cavity, a cancer of the middle ear, a nasopharynx cancer, a ureter cancer, a peritoneal cancer, an omental cancer, a mesentery cancer, or a gastrointestinal carcinoid tumor, or any combination thereof. The target object can comprise an alginate, an alginate derivative, polyethylene glycol (PEG), poly(lactic-co-glycolic acid) (PLGA), poly(ethylene glycol diacrylate) (PEGDA), a collagen, or hyaluronic acid, or any combination thereof. The target object can comprise, for example, a nanoparticle, a nanomaterial, a polymeric biomaterial, a ceramic biomaterial, a metallic biomaterial, or a surface treatment, or any combination thereof. The target object can comprise a surgical device or a portion thereof, for example, a suture. The target object can comprise polypropylene, silk, polyester, polydioxanone, poliglecaprone, polyglactin, or a nylon, or any combination thereof. The target object can be inserted into the microfluidic device using manual techniques, automated techniques, or both. For example, a target object can be inserted using pipetting, injection, or use of one or more surgical tools.

The target can comprise one or more coatings. The one or more coatings of the target can be distinct from the matrix. The one or more coatings of the target can differ with respect to a material composition, a thickness, or patterning, or a combination thereof. Coating and other surface modification characteristics described herein for channel surfaces can be applied to the target and vice versa. The one or more coatings of the target can differ or be the same as one or more coatings of one or more channel surfaces with respect to a material composition, a thickness, or patterning, or a combination thereof. The target can be coated with a plurality of different materials. The target can be coated with a plurality of coatings. The plurality of coatings can differ in material composition, thickness, or patterning, or a combination thereof. The plurality of coating can comprise a first coating comprising a first material adjacent the target and a second coating comprising a second material overlaying the first coating. The target can be coated prior to, during, or after placement in the microfluidic device. Coating of the target can be partial or complete. At least 10%, at least 25%, at least 40%, at least 50%, at least 60%, at least 75%, at least 90%, at least 95%, or at least 99%, or any intervening percentage, or any percentage range therebetween of a target surface can be coated or otherwise modified.

The matrix can comprise a hydrogel. The matrix can comprise a decellularized extracellular matrix, a solubilized basement membrane preparation, a mixture of extracellular matrix proteins, or a tumor derived composition, or any combination thereof. For example, the matrix can comprise solubilized basement membrane matrix secreted by Engelbreth-Holm-Swarm mouse sarcoma cells; the matrix can comprise MATRIGEL (Corning). The matrix can comprise a natural polymer, an artificial polymer, or a cross-linked polymer, or any combination thereof. The matrix can comprise a polysaccharide, a polypeptide, a lipid, a polynucleotide, a hyaluronic acid, a hyaluronic acid methacrylate, a hyaluronic acid diacrylate, a fibronectin, a fibrinogen, a collagen, or a methacrylate collagen, or any combination thereof.

Components described as present in one biological composition can also be present in another biological composition or other biological compositions. The first biological composition can comprise, for example, a fibroblast, or an endothelial cell, or both. Cells that can be used include, for example, B16-F10 cells, MDA-MB-231 cells, MCF-7 cells, HEPG2 cells, or the like, or any combination thereof. Cells can include immune cells, RAW264.7 macrophages, L929 fibroblasts, primary cells, dermal fibroblasts, peripheral blood mononuclear cells, pathogenic bacterial cells, or non-pathogenic bacterial cells, or any combination thereof. Components can comprise viral particles. The cells can be introduced into the microfluidic device, for example, in the form of a suspension of cells within cell culture media, a buffer, a three-dimensional gel, or a pre-gel solution, or any combination thereof. Pharmaceutical drugs or therapeutic materials (small-molecule drugs, large-molecule drugs, antibodies, proteins, nanoparticles, or lipids) can be introduced into the microfluidic device or incorporated within the biomaterials or hydrogels placed within the microfluidic device. Introduced cells can comprise healthy cells, or diseased cells, or both. Cells can be from a human cell line, or a non-human cell line, or both. Cells can be from primary cell culture, or organ explants, or the like. Components added to the microfluidic device can comprise, for example, an anti-inflammatory drug, an anti-fibrotic drug, steroids, or an immune-related drug, or any combination thereof.

The first biological composition can comprise a blood serum, an ascorbic acid, a retinoic acid, proline, bleomycin, cyclophosphamide, amiodarone, procainamide, penicillamine, gold, or nitrofurantoin, or any combination thereof. The first biological composition can comprise a compound, for example, a drug associated with causing fibrosis, for example, lung fibrosis, or liver fibrosis, or both. The first biological composition can comprise an environmental substance, for example, asbestos, silica, titania, zirconia, graphite, coal dust, a mineral dust, or any combination thereof. The first biological composition can comprise a bacterium, a fungus, a component thereof, or a combination thereof. The first biological composition can comprise a biomolecule promoting fibrosis, a complement protein, a blood clotting associated protein, a bacterial cell wall protein, or a lipopolysaccharide (LPS), or any combination thereof. The second biological composition can comprise at least one type of biological cell capable of promoting fibrosis. The second biological composition can comprise a factor that promotes differentiation of a biological cell into a type of biological cell capable of promoting fibrosis, a factor that promotes expression of a biomolecule that promotes fibrosis, or a factor that promotes expansion of a type of biological cell capable of promoting fibrosis, or any combination thereof. The second biological composition can comprise blood serum, a platelet, a white blood cell, a macrophage, a monocyte, a peripheral blood mononuclear cell (PBMC), a stem cell, a mast cell, a dendritic cell, a neutrophil, a granulocyte, a T cell, or a B cell, or any combination thereof. The third biological composition can comprise a population of molecules capable of forming a gradient radiating from the third channel into the first channel, or into both the first and second channels. The third biological composition can comprise a cytokine, a chemokine, a cell nutrient, a transcription factor, or a growth factor, or any combination thereof. The third biological composition can comprise, for example, CCL-2/MCP-1, CCL3, CCL11, fibroblast growth factor (FGF), an interferon, IFN-gamma, an interleukin (IL), IL-4, IL-1, IL-2, IL-5, IL-6, IL-7, IL-9, IL-13, IL-17A, IL-17F, IL-21, IL-22, IL-25, platelet derived growth factor (PDGF), transforming growth factor (TGF)-alpha, transforming growth factor (TGF)-beta, granulocyte colony-stimulating factor (G-CSF), macrophage colony stimulating growth factor (M-CSF), granulocyte macrophage colony-stimulating factor (GM-CSF), prothrombin, or thrombin, or any combination thereof. The at least one type of molecule of the gradient can comprise a cytokine, a chemokine, a cell nutrient, a transcription factor, or a growth factor, or any combination thereof. The at least one type of molecule of the gradient can comprise a transforming growth factor beta (TGF-beta). The TGF-beta can comprise TGF-beta1, TGF-beta2, or TGF-beta3, or any combination thereof. The first biological composition, the second biological composition, or the third biological composition, or any combination thereof can comprise a labeled cell, or a labeled biomolecule, or both. The first biological composition, the second biological composition, or the third biological composition, or any combination thereof can comprise a compound having known or potential anti-fibrotic efficacy.

The fibrotic diagnostic system can be used to model one or more kinds of fibrosis. Fibrosis can be associated with any tissue or organ, for example, the lungs, the heart, the liver, the pancreas, the kidneys, the bladder, the skin, skeletal muscle, or smooth muscle, or any combination thereof. Examples of fibrosis can include endomyocardial fibrosis, cystic pancreatic fibrosis, diatomite fibrosis, diffuse interstitial fibrosis, idiopathic fibrosis, endomyocardial fibrosis, graphite fibrosis, idiopathic pulmonary fibrosis, idiopathic retroperitoneal fibrosis, mediastinal fibrosis, neoplastic fibrosis, nodular subepidermal fibrosis, panmural bladder fibrosis, periureteric fibrosis, postfibrinous fibrosis, proliferative fibrosis, pulmonary fibrosis, replacement fibrosis, retroperitoneal fibrosis, or root sleeve fibrosis, or any combination thereof. The fibrosis can be the result of a tissue autograft, a tissue allograft, or a tissue xenograft, or any combination thereof. The fibrosis can be the result of an inorganic material, for example, a metal, a ceramic, a mineral, or any combination thereof. Fibrosis can be characterized by deposition of collagen on the target object. One or more types of collagen can characterize the fibrosis. The collagen can comprise, for example, collagen I, collagen II, collagen III, collagen IV, collagen V, collagen, VI, collage, collagen VII, collagen VIII, collagen IX, collagen X, collagen XI, collagen XII, collagen XIII, collagen XIV, collagen XV, collagen XVI, collagen XVII, collagen XVIII, collagen XIX, collagen XX, collagen XXI, collagen XXII, collagen XXIII, collagen XXIV, collagen XXV, collagen XXVI, collagen XXVII, or collagen XXVIII, or any combination thereof. One or more types of procollagen, or collagen metabolites, or both can characterize the fibrosis.

The fibrotic diagnostic system can be preloaded with one or more components such as reagents, or a target object, or both. For example, the fibrotic diagnostic system can comprise the matrix, the first biological composition, the second biological composition, or the third biological composition, or any combination thereof. The fibrotic diagnostic system can comprise the target object. The fibrotic diagnostic system can comprise a gradient of at least one molecule across the first channel, the second channel, or the third channel, or any combination thereof. One or more components of the matrix, the first biological composition, the second biological composition, or the third biological composition, or any combination thereof can be anchored to a channel surface.

The fibrotic diagnostic system can comprise a detector. The detector can comprise a sensor. The sensor can be configured to detect fibrosis or characteristic indicative of the same of the target object. The detector can be configured to receive the microfluidic device, for example, in a receptacle or on a stage of a microscope. The detector can comprise a detector housing comprising a detector interior. The detector can comprise a receptacle configured to receive the microfluidic device. For example, the receptacle can comprise an opening in the detector housing. Any number, or any kind, or both of receptacle can be employed. A receptacle can be configured to receive one or more microfluidic devices. A plurality of receptacles can be provided to receive a corresponding plurality of microfluidic devices.

The fibrotic diagnostic system can comprise a thermal subsystem. The thermal subsystem can comprise a heater, or a cooler, or both. The thermal subsystem can be configured to regulate a temperature of the microfluidic device. The detector can comprise the thermal subsystem. The thermal subsystem can be configured to regulate the temperature of the microfluidic device, for example, when it is in the receptacle.

The fibrotic diagnostic system can comprise a fluidic subsystem configured for fluidic communication with the microfluidic device. The fluidic subsystem can be configured to load one or more components into the microfluidic device. The fluidic subsystem can be configured to supply a solid, a liquid, or a gas, or any combination thereof to the microfluidic device. The fluidic subsystem can be configured to load the target object, the matrix, the first biological composition, the second biological composition, or the third biological composition, or any combination thereof into the microfluidic device. The fluidic subsystem can be configured to supply one or more nutrients to the microfluidic device. The fluidic subsystem can be configured to regulate a pressure in an interior the microfluidic device, for example in one or more of the channels. A pressure of an interior of the microfluidic device, for example, a channel, can be the same, above, or below atmospheric pressure. A pressure differential can be applied between channels, within a channel, or both. A pressure at, above, or below an osmotic pressure can be applied. The fluidic subsystem can be configured to regulate a gradient across the first channel, the second channel, or the third channel, or any combination thereof. The gradient can be controlled, for example, by adding a gradient solute/molecule, or applying a pressure, or both. A pressure can be applied, for example, at, above, or below a osmotic pressure associated with a gradient molecule. The fluidic subsystem can be configured to remove one or more component from the microfluidic device. The detector, for example, can comprise the fluidic subsystem. The fluidic subsystem can be configured for fluidic communication with the microfluidic device, for example, when the microfluidic device is in the receptacle. The fluidic subsystem can comprise a channel, a valve, a manifold, or a pump, or any combination thereof. The fluidic subsystem can comprise a handheld pipet. The fluidic subsystem can be configured to provide a desired flow rate of a solid, a liquid, or a gas, or any combination thereof through one or more of the plurality of channels. A flow rate can be, for example, less than about 0.001 μL/s, from about 0.001 μL/s to about 100 mL/s, from about 0.01 L/s to about 10 mL/s, from about 0.1 μL/s to about 1.0 mL/s, from about 1.0 μL/s to about 100 μL/s, from about 10 μL/s to about 50 μL/s, or greater than 100 mL/s, or any value there between, or any intervening range. The fluidic subsystem can comprise or be configured to fluidically connect to supplies of one or more reagents, for example, a plurality of reservoirs or a single reservoir.

The sensor can be configured to detect a particle, or a wave, or both emitted from the target object, or the microfluidic device, or both indicative of fibrosis. The sensor can be configured to detect electromagnetic radiation. The electromagnetic radiation can comprise, for example, ultraviolet radiation, visible light, or infrared radiation, or any combination thereof. The electromagnetic radiation can comprise fluorescence, or phosphorescence, or both. The detector can comprise a particle source, or a wave source, or both configured to transmit a particle, a wave, or both to the target object, or the microfluidic device, or both. The fibrotic diagnostic system can comprise a source of electromagnetic radiation. For example, the detector can comprise the source of electromagnetic radiation. The source of electromagnetic radiation can comprise, for example, an light emitting diode, a laser, an incandescent light source, an arc light source, a halogen light source, a chemical vapor light source, or a fluorescent light source, or any combination thereof. The detector can comprise an optical subsystem configured to pass radiation, or affect radiation, or both. The optical subsystem can comprise one or more lenses, one or more filters, one or more mirrors, or one or more diffraction gratings, or any combination thereof. The optical subsystem, or the sensor, or both can comprise a microscope or one or more components thereof. The optical subsystem can comprise the sensor, or the source of electromagnetic radiation, or both. The sensor can comprise a microphone and the fibrotic diagnostic system can comprise a source of sound waves. The sound waves can comprise ultrasound and the microphone can detect ultrasound. The sensor can comprise a force sensor, an actuator, or both. Fibrosis of the target object can result in a fibrotic structure having a tensile strength, or a compressive strength, or both that can be detected by the sensor.

The sensor can be configured to generate an output signal based on an input signal detected by the sensor. The detector can comprise a transmitter, a receiver, a transceiver, or any combination thereof configured to receive information, or send information, or both. The information can comprise the output signal. The information can comprise an operational signal for operation of the sensor or other component of the system. The information can travel via a network. The network can be a local or long-distance network. The network can be wired, or wireless, or both. The detector or other component of the system can comprise a data port. Any desired number of data ports can be provided. The system can further comprise a processor configured to receive and process information. The processor can be configured to control one or more subsystems or components of the fibrotic diagnostic system. For example, the processor can be configured to control the sensor, the source of electromagnetic radiation, the optical subsystem, the thermal subsystem, or the fluidic subsystem, or any combination thereof. The processor can be configured to receive the output the signal from the sensor, to receive an operational signal from a user input, or both. The detector, for example, can comprise the processor. The processor can comprise a local processor, or a remote processor, or both. The processor can be a microprocessor. The processor can be a central processing unit (CPU). The processor can comprise one or more processing chips. The system can comprise a memory configured for communication with the processor. The memory can be local or remote. The memory can be transitory, or non-transitory, or both. The memory can comprise a random access memory (RAM), or a read only memory (ROM), or both. The memory can be configured to store a program that controls the fibrotic diagnostic system or a component thereof. The processor, for example, can comprise the memory. The detector, for example, can comprise the memory.

The fibrotic diagnostic system can comprise a user interface. The system can comprise any number of user interfaces, or any kind of user interface, or both. A user interface can be interactive, or non-interactive, or both, for example, a user interface can comprise one or more components that are interactive and one or more components that are not interactive. A user interface can be free-standing or integrated into one or more system subsystem or component. The user interface can comprise a monitor, a button, a keyboard, a mouse, a light signal, a camera, an eye piece lens, a speaker, or a microphone, or any combination thereof. The monitor can comprise, for example, a touchscreen. The camera can be a still photographic camera, or a video camera, or both. The eye piece lens can be part of a microscope system or subsystem. Two eye piece lenses can be provided, for example, to allow for binocular viewing. The detector, for example, can comprise the user interface.

The fibrotic diagnostic system can comprise a power source, or a power interface, or both. The power source can comprise a battery, for example, a rechargeable battery. The power interface, for example, can comprise an inverter for receiving alternating wall current and outputting direct current. The detector, for example, can comprise the power source, or the power interface, or both.

The fibrotic diagnostic system can comprise or be provided as a kit. The kit can comprise two or more components of the system. The kit can comprise a supply of multiples of a particular system component, for example, multiple multifluidic devices. The kit can comprise, for example, the microfluidic device and one or more reagents for use in the microfluidic device. The kit can comprise, for example, the microfluidic device; and the matrix, the first biological composition, the second biological composition, or the third biological composition, or any combination thereof. One or more components or subsystems of the fibrotic diagnostic system, whether or not supplied as a kit, can comprise an identifier. The identifier can comprise a serial number, a product number, a bar code, a RFID tag, a color code, a name, a number, or an alphanumeric code, or any combination thereof.

The disclosure provides methods for using the fibrotic diagnostic systems, subsystems thereof, and components thereof. For example, a method of analyzing an target object for susceptibility to fibrosis is provided. The method can comprise one or more of the following steps. The target object, the matrix, and the first biological composition can be placed in the first channel of the microfluidic device. The second biological composition can be placed in the second channel of the microfluidic device. The third biological composition can be placed in the third channel of the microfluidic device. The target object, or the first channel, or both can be analyzed to determine the presence or absence of fibrosis associated with the target object.

The analyzing can be performed after a predetermined period following placement of the target object, the matrix, the first biological composition, the second biological composition, or the third biological composition, or any combination thereof into the microfluidic device. The predetermined period can be sufficient to allow formation of the gradient of at least one type of molecule. The analyzing can be performed two or more times. Any two consecutive analyses can be separated by a predetermined period. A period can be counted as seconds, minutes, hours, days, or weeks. A period can be, for example, less than about a minute, from about one minute to about month, from about one hour to about two weeks, from about three hours to about one week, from about six hours to about three days, from about 12 hours to about one day, more than about a month, or any period therebetween, or a period range therein. The analyzing can comprise removing one or more components from the microfluidic device.

The analyzing can comprise an immunoassay. The analyzing can comprise use of an antibody. The antibody can be labeled with a detectable label. The analyzing can comprise application of a primary antibody and a secondary antibody that can comprise a detectable label and binds to the primary antibody. The primary antibody can bind a collagen, an alpha-smooth muscle actin, or both. The analyzing can comprise a histological assay. Cells and/or tissues can be fixed for assaying, for example, prior to staining. Any suitable fixative can be used, for example a formaldehyde fixative, neutral buffered formalin (NBF), paraformaldehyde (PFA), or methanol. The fixative can be an aldehyde fixative, a mercurial fixative, an alcohol fixative, an oxidizing agent fixative, or a picrate fixative, or any combination thereof, Further examples of fixatives are acetone, phosphate buffered formalin, zinc formalin, formal calcium, formal saline, alcoholic formalin, formol acetic alcohol, zinc formalin, Alcian Blue, Bouin's solution, B-5 fixative, Carnoy's solution, Clarke's solution, Gendre's solution, Helly's fixative, Hollande's solution, methacarn, and Zenker's fixative. Cells and/or tissues can be partially or fully dehydrated for assaying. The analyzing can comprise embedding, or antigen retrieval, or both. Any suitable stain or combination of stains can be used. The stain can comprise a dye. Picric acid can be used. The analyzing can comprise, for example, application of picrosirius red dye alone or in combination with one or more additional stains. Example of stains include carmine, hematin, hematoxylin, fluorescein, eosin, silver nitrate, Giemsa stains, Golgi stain, Gram stain, Kluver-Barrera stain, Mallory's CT stains, Masson's stain, periodic acid-Schiff (PAS) stain, Romanowsky stains, trichrome stains, AZAN trichrome stain, toluidine blue, and Verhoeff's Van Gieson stain, and any combination thereof.

The analyzing can comprise visually inspecting the target object, or the first channel, or both for one or more indications of fibrosis associated with the target object. The analyzing can comprise using microscopy. The analyzing can comprise using one or more spectroscopic techniques. For example, the analyzing can comprise nonlinear optical (NLO) multi-photon microscopy, second harmonic generation (SHG) imaging, third harmonic generation (THG) imaging, two-photon excited fluorescence (TPEF), two-photon excited fluorescence (TPEF), cell tracker dye imaging, visible light imaging, (ELISA), a Griess assay, histopathology, immunostaining, cleared tissue microscopy, or proteome profiling of homogenized hydrogel, or any combination thereof. The analyzing can comprise using the detector to detect one or more indications of fibrosis associated with the target object. The analyzing can comprise inserting the microfluidic device into the receptacle.

The method can comprise preparing the target object, the matrix, the first biological composition, the second biological composition, or the third biological composition, or any combination thereof from a patient sample. The method can comprise implanting the target object into or onto a patient. For example, the method can comprise implanting a medical device, or a tissue, or both into or onto a patient, wherein the target object is a representative model of the medical device, or the tissue, or both. The method can comprise removing a medical device, or a tissue, or both from a patient, wherein the target object is a representative model of the medical device, or the tissue, or both.

The method can comprise administering an anti-fibrosis agent to the patient. The analyzing can comprise determining a quantitative measure of fibrosis. The dosage of an anti-fibrosis agent administered to the patient can be based on the quantitative measure of fibrosis. The anti-fibrosis agent can be administered to the patient before, concurrently with, or after implanting the target object, a tissue associated with the target object, or a medical device associated with the target object, or any combination thereof. One or more therapeutics can be administered to the patient in addition to or in the alternative to the anti-fibrosis agent. The patient can be, for example, a mammalian patient, or a human patient, or both.

A method of screening a therapy for efficacy of treating fibrosis is provided. The method can comprise one or more of the following steps. The target object, the matrix, and the first biological composition can be placed in the first channel of the microfluidic device. The second biological composition can be placed in the second channel of the microfluidic device. The third biological composition can be placed in the third channel of the microfluidic device. The therapy can be performed in the microfluidic device. The target object, or the first channel, or both can be analyzed to determine a presence, an absence, or a degree of fibrosis associated with the target object. The efficacy of the therapy can be determined based on a difference in the presence, the absence, or the degree of fibrosis without performing the therapy. The therapy can comprise a therapeutic compound and the performing can comprise placing the therapeutic compound in the first channel, the second channel, or the third channel, or any combination thereof. The therapeutic compound can comprise a pharmaceutical compound, or a biologic, or both. The therapy can comprise a modification of the target object. The therapy can comprise placing a medical device in the first channel, the second channel, or the third channel, or any combination thereof. The therapy can comprise applying radiation to the microfluidic device. The method can comprise treating a patient with the screened therapy. Methods of screening provided herein comprise high-throughput screening methods.

A method of screening a therapy for efficacy of treating cancer is provided by the disclosure. The method can comprise one or more of the following steps. The target object, the matrix, and the first biological composition can be placed in the first channel of the microfluidic device, wherein the target object can comprise a neoplastic cell. The second biological composition can be placed in the second channel of the microfluidic device. The third biological composition can be placed in the third channel of the microfluidic device. The therapy can be performed on the microfluidic device. The target object, or the first channel, or both can be analyzed to determine a presence, an absence, or a degree of fibrosis associated with the target object. An efficacy of the therapy can be determined based on a difference in the presence of, in the absence of, or in the degree of, cancer without performing the therapy. Other screening methods employing the fibrotic diagnostic systems are also provided. For example, screening methods can be used to monitor immunological phenomena associated with foreign bodies generally as well as more specific phenomena such as graft versus host disease (GVHD) or autoimmune syndromes.

Non-transitory computer-readable media are provided by the disclosure. For example, a non-transitory computer-readable medium storing a program comprising instructions to perform a method of the disclosure is provided. A non-transitory computer-readable medium storing a program comprising instructions to control the fibrotic diagnostic system or a subsystem, or a component thereof, or any combination thereof is provided.

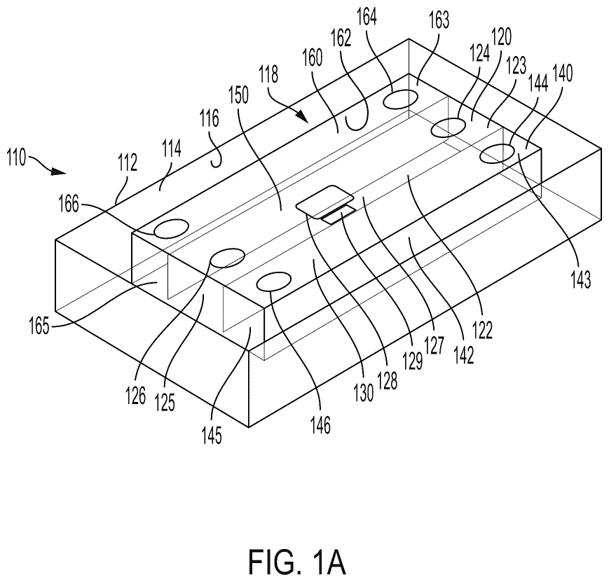

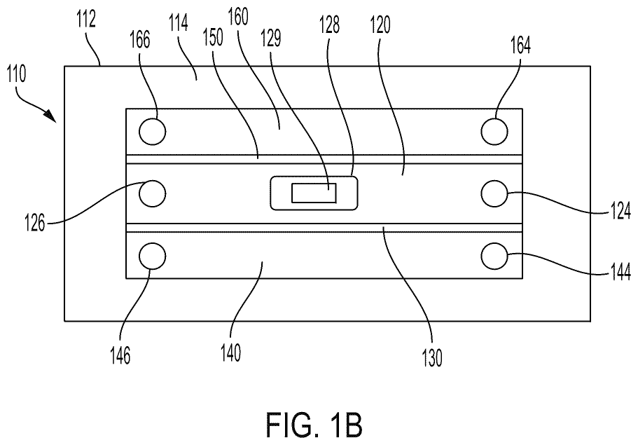

FIG. 1A is a perspective view of a microfluidic device 110 in accordance with the present disclosure. Microfluidic device 110 can be used in a fibrotic diagnostic system, or method, or both of the present disclosure. Microfluidic device 110 can comprise a device housing 112, an outer surface 114 of device housing 112. Device housing 112 can define a device interior 116 that can comprise a plurality of channels 118. Plurality of channels 118 can comprise a first channel 120 between a second channel 140 and a third channel 160. A first septum 130 can separate or otherwise demark first channel 120 from second channel 140. A second septum 150 can separate or otherwise demark first channel 120 from third channel 150. First and second septa 130, 150 can have any design, for example, a design such as a porous membrane or an array of pillars (micropillars) described herein. First channel 120 can comprise an interior 122, a first end 123, a first channel port 124 proximal first end 123, a second end 125, and a second channel port 126 proximal second end 125. First channel 120 can comprise a surface 127 and surface 127 can be modified, for example, with a surface modification 129 configured to receive and hold one or more target object. First channel 120 can comprise a third channel port 118 between first channel port 124 and second channel port 126 to facilitate insertion and retrieval of one or more target objects from interior 122. Second channel 140 can comprise an interior 142, a first end 143, a first channel port 144 proximal first end 143, a second end 145, and a second channel port 146 proximal second end 145. Third channel 160 can comprise an interior 162, a first end 163, a first channel port 164 proximal first end 163, a second end 165, and a second channel port 166 proximal second end 165. Channel ports can provide passage between a channel interior through device housing 112 to device surface 116 and regions otherwise exterior of microfluidic device 110. FIG. 1B is a plan view of the microfluidic device shown in FIG. 1A.

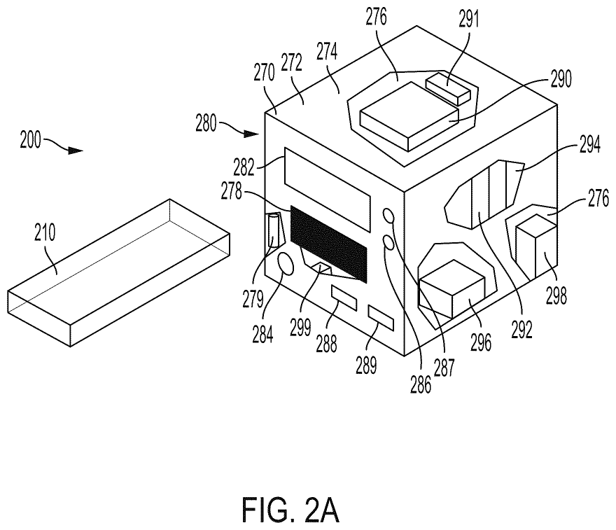

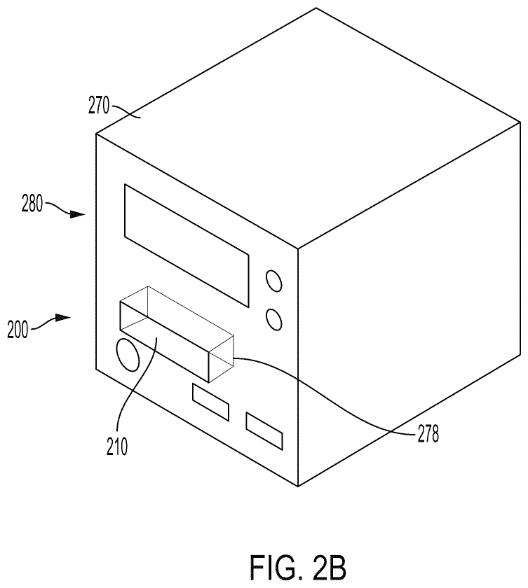

FIG. 2A is a perspective view of a fibrotic diagnostic system 200 showing a microfluidic device 210 disengaged from a detector 270 in accordance with the present disclosure. Microfluidic device 210 can comprise features, for example, described for microfluidic device 110 or any other microfluidic device described herein. Detector 270 can comprise a detector housing 272, a detector surface 274, a detector interior 276, and a receptacle 278 permitting entry of microfluidic device 210 in whole or part into detector interior 276. The depiction of detector 270 is for discussion purposed and can adopt other designs as appropriate for a given embodiment. For example, detector 270 can adopt a more open design such as that of a microscope with an open receptable in the form of a stage for placement of microfluidic device 210. Detector 270 can comprise a user interface 280 and that shown in FIGS. 2A, 2B is for illustrative purposes and is non-limiting. A user interface of system 200 can be integrated into detector 270, or provided as standalone components, or both. User interface 280 is shown comprising such elements as a monitor 282, a button 284, signals 286, 287, and data ports 288, 289. Screen 282 can comprise, for example, a screen. The screen can be provided as a touchscreen. The screen can display a graphic user interface (GUI). Button 284 can, for example, allow for control of one or more function of detector 270, for example, to power on/off detector 270, or initiate imaging of microfluidic device 210, or both. A single button is shown, but any number of buttons can be provided or omitted, for example, a button for respective functions, or a plurality of buttons in the form of a keyboard or keypad. A button or buttons can be replaced with other types of actuators such as a toggle switch. Signals 286, 287 can comprise lights, for example, lights of respective colors. Respective signals can correspond, for example, to particular operational states of detector 270, for example a power state, engagement of microfluidic device 210, or imaging of microfluidic device 210, or any combination thereof. Alternatively or additionally, a signal can change colors corresponding to a particular operational state. Two signals are shown but any number of buttons can be provided or omitted. One or more signals can adopt the form of a speaker, or a microphone, or both. Data ports 288, 289 can provided for any number of different functionalities, for example, data transfer, power transfer, and connection to peripheral user interface components. Two data ports are shown, but any number of data ports can be provided or omitted. In embodiments comprising a microscope, user interface 280 can comprise one or more eye pieces configured for viewing of microfluidic device 210 by a user.

FIG. 2A contains several illustrative break-aways in detector housing 272 in order to show various components that can be located in detector interior 276. For example, a fluidics subsystem 279 is shown that can be provided to supply microfluidic device 210 with various components when held in receptacle 278. A thermal subsystem 299, for example, a heater, is shown that can be provided to regulate a temperature of microfluidic device 210 when held in receptacle 278. Sensor 290 is shown that can detect one or more signals from microfluidic device 210, for example, an emission radiation resulting from an excitation radiation emanating from radiation source 291. The number and placement of sensors and light sources can be varied. An optical subsystem 292 is also shown and can comprise one or more optical elements such as lenses, mirrors, diffraction gratings, and filters. Optical subsystem 292 can comprise one or both of sensor 290 and radiation source 291. A processor 294 is shown that can process information related to operation of detector 270. A transceiver 296 is shown that can receive and transmit signals, for example, to/from user interface 280, sensor 290, processor 294, a network via data ports 288, 289, or network wirelessly, or any combination thereof. A power source 298 is shown that can take the form of a standalone power source, or a power interface for transmitting/transforming power from an external source, or both. FIG. 2B is a perspective view of fibrotic diagnostic system 200 of FIG. 2A showing microfluidic device 210 engaged with detector 270 through receptacle 278 in accordance with the present disclosure.

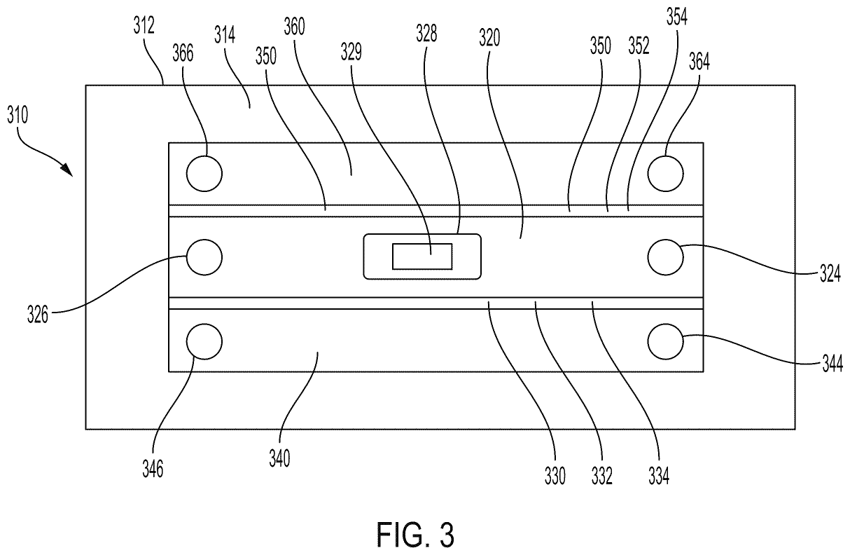

FIG. 3 is a plan view of a microfluidic device 310 in accordance with the present disclosure. Microfluidic device 310 can be used in a fibrotic diagnostic system, or method, or both of the present disclosure. Microfluidic device 310 can comprise a device housing 312, an outer surface 314 of device housing 112. Device housing 312 can define a device interior 316 that can comprise a plurality of channels. The plurality of channels can comprise a first channel 320 between a second channel 340 and a third channel 360. A first septum 330 can separate or otherwise demark first channel 320 from second channel 340. First septum 330 is shown having a micropillar design comprising a plurality of pillars (micropillars) 332 defining a plurality of openings 334 therebetween. A second septum 350 can separate or otherwise demark first channel 320 from third channel 350. Second septum 350 is shown having a micropillar design comprising a plurality of pillars (micropillars) 352 defining a plurality of openings 354 therebetween. The plurality of channels can comprise features similar to or the same as those shown for plurality of channels 118 in FIG. 1A. For example, first channel 320 can comprise a first channel port 324 proximal a first end, a second channel port 326 proximal a second end, and a third channel port 318 between first channel port 324 and second channel port 326 to facilitate insertion and retrieval of one or more target objects from a container 329 in first channel 320. Second channel 340 can comprise a first channel port 344 proximal a first end and a second channel port 346 proximal a second end. Third channel 360 can comprise a first channel port 364 proximal a first end and a second channel port 366 proximal a second end. Channel ports can provide passage between a channel interior through device housing 312 to device surface 316 and regions otherwise exterior of microfluidic device 310.

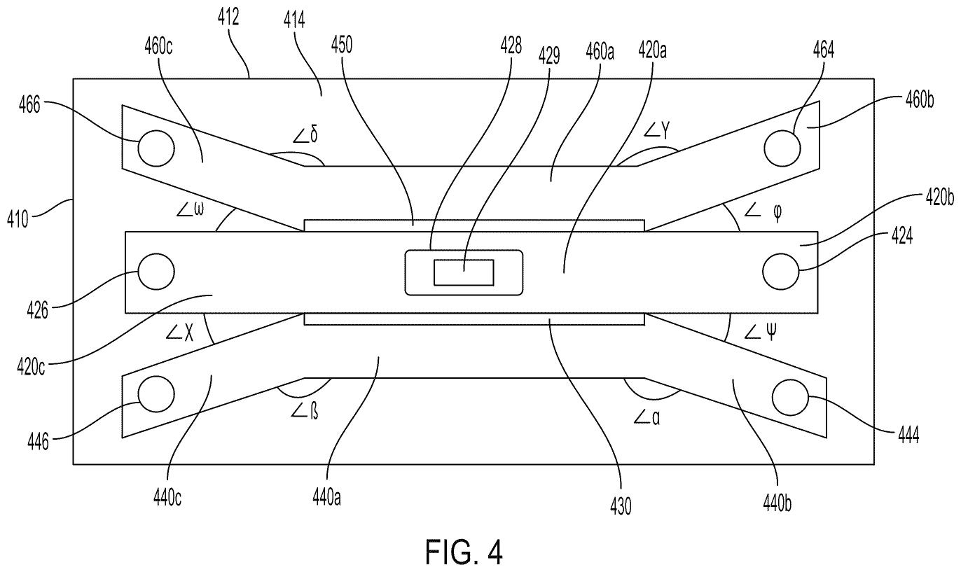

FIG. 4 is a plan view of a microfluidic device 410 in accordance with the present disclosure. Microfluidic device 410 can be used in a fibrotic diagnostic system, or method, or both of the present disclosure. Microfluidic device 410 can comprise a device housing 412, an outer surface 414 of device housing 412. Device housing 412 can define a device interior 416 that can comprise a plurality of channels. The plurality of channels can comprise a first channel 420 between a second channel 440 and a third channel 460. A first septum 430 can separate or otherwise demark first channel 420 from second channel 440. A second septum 450 can separate or otherwise demark first channel 420 from third channel 450. The plurality of channels can comprise features similar to or the same as those shown for plurality of channels 118 in FIG. 1A. Further, one or more of the channels can be divided into channel segments with respective segments differing from one another in one or more parameter. For example, first channel 420 can comprise a first channel port 424 proximal a first end, a second channel port 326 proximal a second end, and a third channel port 418 between first channel port 424 and second channel port 426 to facilitate insertion and retrieval of one or more target objects from a container 429 in first channel 420. First channel 420 can have three segments 420a, 420b, and 420c. First channel port 424 can be located in second segment 420b, second channel port 426 can be located in third segment 420c, and third channel port can be located in first segment 420a. First segment 420a can be adjacent to both septa 430, 450 along its length. Second channel 440 can comprise a first channel port 444 proximal a first end and a second channel port 446 proximal a second end. Second channel 440 can have three segments 440a, 440b, and 440c with first channel port 444 located in second segment 440b, second channel port 446 located in third segment 440c. Second segment 440b can have an angle alpha with respect to first segment 440a. Third segment 440c can have an angle beta with respect to first segment 440a. Angles alpha and beta can be the same or different. They can be acute, right, or obtuse angles. Second segment 440b can have an angle psi with respect to second segment 420b. Third segment 440c can have an angle chi with respect to third segment 420c. Angles psi and chi can be the same or different. They can be acute, right, or obtuse angles. Third channel 460 can comprise a first channel port 464 proximal a first end and a second channel port 466 proximal a second end. Third channel 440 can have three segments 460a, 460b, and 460c with first channel port 464 located in second segment 460b, second channel port 466 located in third segment 460c. Second segment 460b can have an angle gamma with respect to first segment 460a. Third segment 460c can have an angle delta with respect to first segment 460a. Angles gamma and delta can be the same or different. They can be the same or different as angles alpha and beta. They can be acute, right, or obtuse angles. Second segment 460b can have an angle phi with respect to second segment 420b. Third segment 460c can have an angle omega with respect to third segment 420c. Angles phi and omega can be the same or different. They can be the same or different as angles psi and chi. They can be acute, right, or obtuse angles. Channel ports can provide passage between a channel interior through device housing 412 to device surface 416 and regions otherwise exterior of microfluidic device 410.

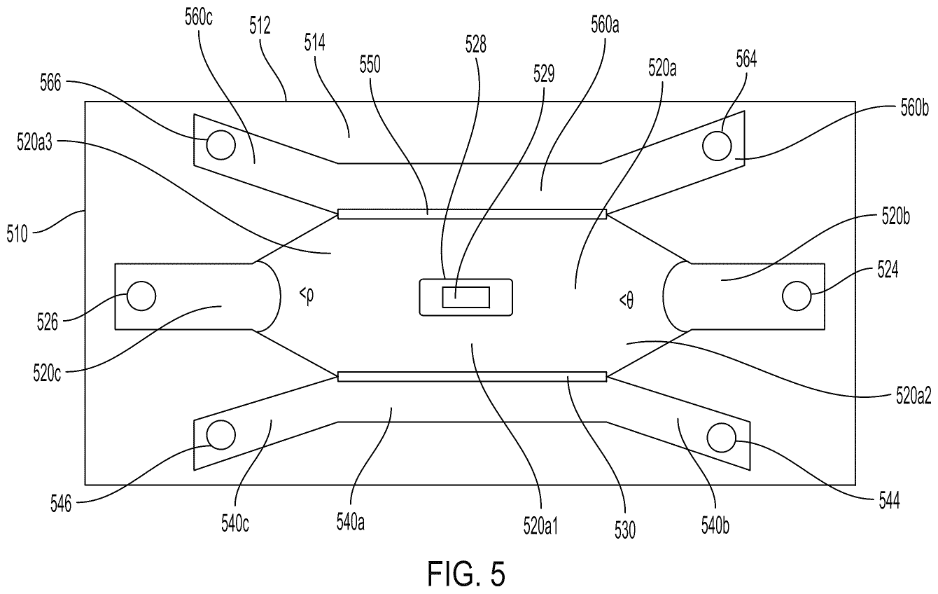

FIG. 5 is a plan view of a microfluidic device 510 in accordance with the present disclosure. Microfluidic device 510 can be used in a fibrotic diagnostic system, or method, or both of the present disclosure. Microfluidic device 510 can comprise a device housing 512, an outer surface 514 of device housing 512. Device housing 512 can define a device interior 516 that can comprise a plurality of channels. The plurality of channels can comprise a first channel 520 between a second channel 540 and a third channel 560. A first septum 530 can separate or otherwise demark first channel 520 from second channel 540. A second septum 550 can separate or otherwise demark first channel 520 from third channel 550. The plurality of channels can comprise features similar to or the same as those shown for plurality of channels 118 in FIG. 1A. Further, one or more of the channels can be divided into channel segments with respective segments differing from one another in one or more parameter. For example, first channel 520 can comprise a first channel port 424 proximal a first end, a second channel port 526 proximal a second end, and a third channel port 518 between first channel port 524 and second channel port 526 to facilitate insertion and retrieval of one or more target objects from a container 529 in first channel 520. First channel 520 can have three segments 520a, 520b, and 520c. First segment 520a can comprise three regions 520a1, 520a2, and 520a3. First channel port 524 can be located in second segment 520b, second channel port 526 can be located in third segment 520c, and third channel port can be located in first segment 520a. First region 520a1 can be adjacent to both septa 530, 550 along its length. Region 520a2 can be between region 520a1 and second segment 520b. Region 520a3 can be between region 520a1 and third segment 520c. Regions 520a2 and 520a3 can vary with respect to cross-sectional area, width, or diameter, or any combination thereof along their respective lengths. For example, regions 520a2 and 520a3 can be defined by angles theta and rho respectively. Angles theta and rho can be the same or different. They can be acute, right, or obtuse. Second channel 540 can comprise a first channel port 544 proximal a first end and a second channel port 546 proximal a second end. Second channel 540 can have three segments 540a, 540b, and 540c with first channel port 544 located in second segment 440b, second channel port 546 located in third segment 540c. Third channel 560 can comprise a first channel port 564 proximal a first end and a second channel port 566 proximal a second end. Third channel 540 can have three segments 560a, 560b, and 560c with first channel port 564 located in second segment 560b, second channel port 566 located in third segment 560c. Channel ports can provide passage between a channel interior through device housing 512 to device surface 516 and regions otherwise exterior of microfluidic device 510.

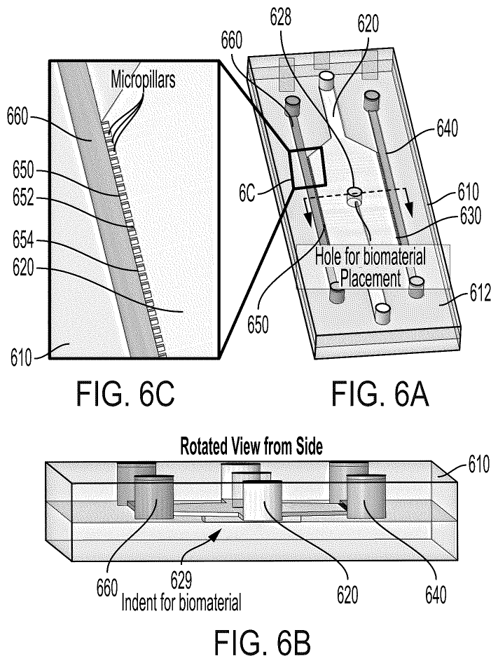

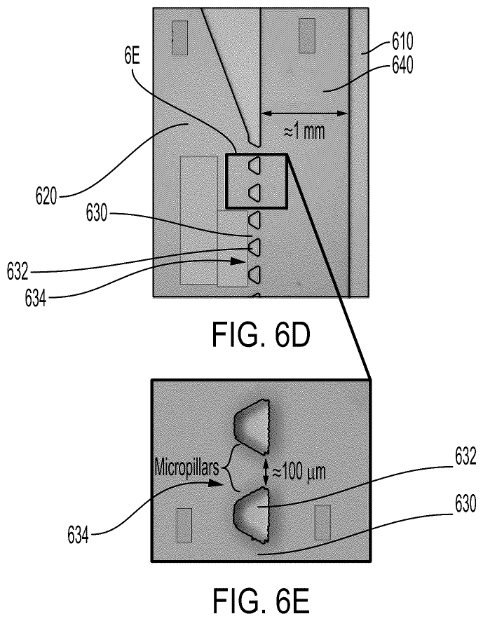

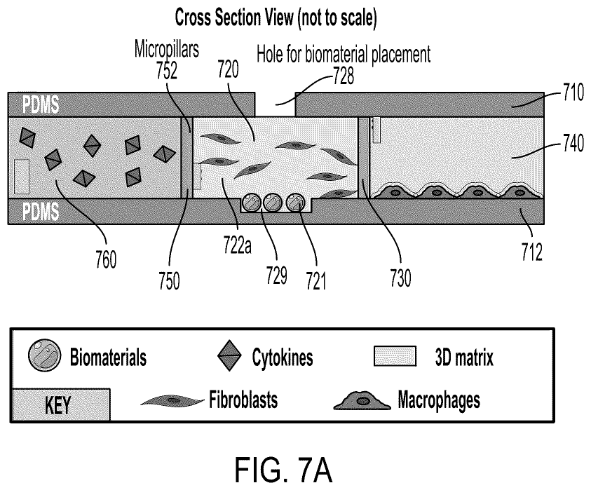

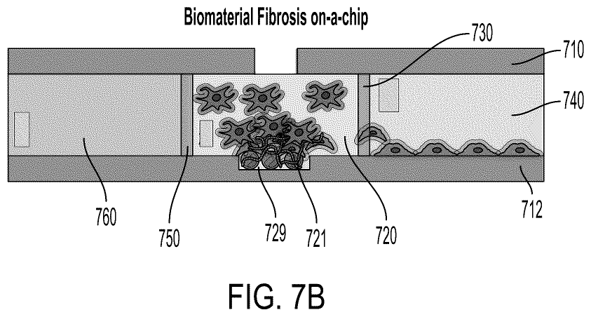

FIG. 6A is a top perspective view of a microfluidic device 610 in accordance with the present disclosure. FIG. 6B is a front perspective view of microfluidic device 610 shown in FIG. 6A. FIG. 6C is an enlarged perspective view of a portion of microfluidic device 610 shown in FIG. 6A. FIG. 6D is an enlarged plan view of a portion of microfluidic device 610 shown in FIG. 6A. FIG. 6E is a further enlarged plan view of a portion of the enlarged view shown in FIG. 6D. FIGS. 6A-6E are discussed in detail below in Example 1. FIG. 7A is a schematic cross-sectional view of a loaded microfluidic device 710 at a first time point in accordance with the present disclosure. FIG. 7B is a schematic cross-sectional view of loaded microfluidic device 710 shown in FIG. 7A at a second time point. FIGS. 7A and 7B are discussed in detail below in Example 1.

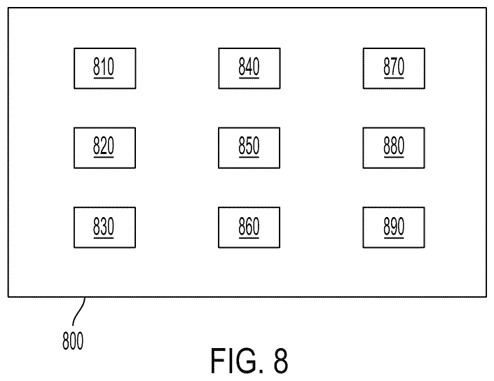

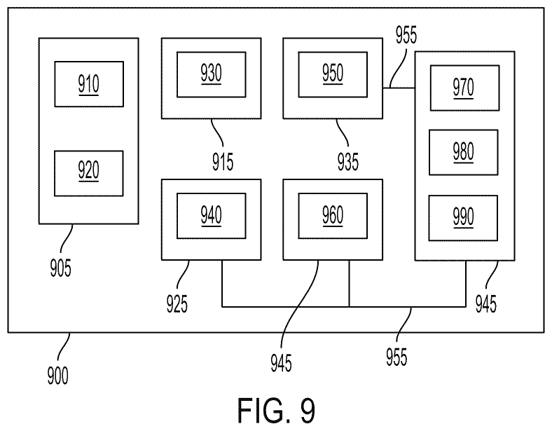

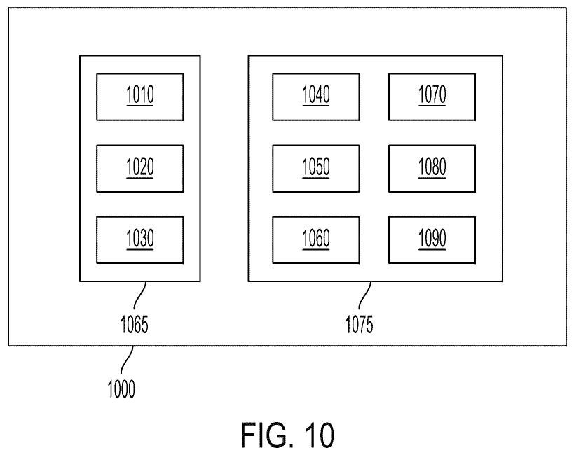

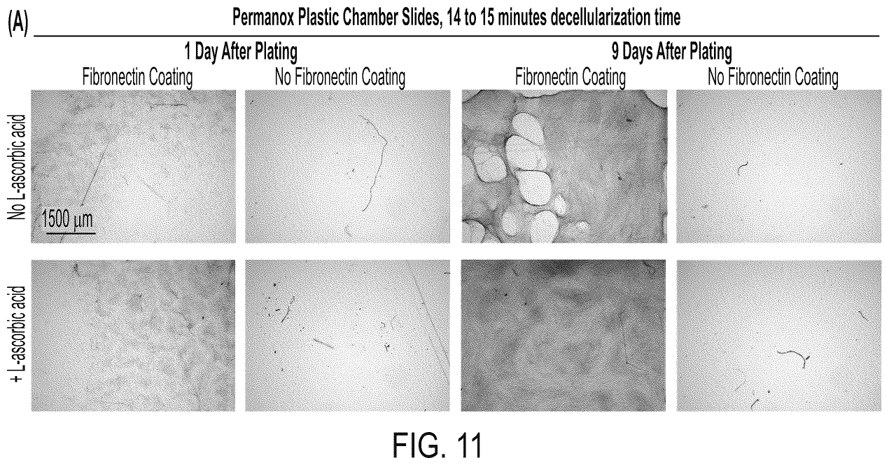

FIG. 8 is a schematic diagram of a fibrotic diagnostic system 800 in accordance with the present disclosure. Fibrotic diagnostic system 800 can comprise various components and subsystems, for example, a microfluidic device 810, reagents 820, a target object 830, a fluidic subsystem 840, a thermal subsystem 850, a sensor 860, a processor 870, a memory 880, and a user interface 890. These various components and subsystems can be arranged, configured, combined, multiplied, or omitted, or any combination thereof without departing from the present disclosure. One or more components or subsystems of the system can be provided in one or more sterile enclosure or other enclosure separating its contents from the surrounding environment. FIGS. 9 and 10 depict two examples of such systems.

FIG. 9 is a schematic diagram of a fibrotic diagnostic system 900 in accordance with the present disclosure. In system 900, a kit 905 is provided comprising a microfluidic device 910 and reagents 920. A target object preparation subsystem 915 is provided that is configured to form a target object 930 for insertion into microfluidic device 910 along with reagents 920. A microfluidic device loader 925 is provided that can comprise a microfluidics subsystem for loading reagents 920, or target object 930, or both into microfluidic device 910. An incubator 935 is provided that can comprise a thermal subsystem 950 configured to regulate a temperature of microfluidic device 910 and its contents during a fibrotic diagnostic assay. A detector 945 that comprises a sensor 960 is provided for imaging microfluidic device 910, or its contents, or both during a fibrotic diagnostic assay. A computing device 945 is provided that can comprise a processor 970, a memory 980, and a user interface 990 that can be used to control system 900 and process information relevant to the same. One or more system components or subsystems can be interconnected through a network 955.

FIG. 10 is a schematic diagram of a fibrotic diagnostic system 1000 in accordance with the present disclosure. The system can comprise a loaded microfluidic device 1065 comprising a microfluidic device 1010 containing reagents 1020 and a target object 1030. The system can comprise a detector 1075 comprising a fluidics subsystem 1040, a thermal subsystem 1050, a sensor 1060, a processor, 1070, a memory 1080, and a user interface 1075. After a time elapsed from the loading of microfluidic device 1010, loaded microfluidic device 1065 or a component thereof can be inserted into or onto detector 1075 for analyzing possible fibrosis of target object 1030.

### Example 1

A three-channel microfluidic device 610, 710 (FIGS. 6A-6E, 7A, and 7B) for use in the co-culture of fibroblasts and macrophages with a target object (721), for example, three-dimensional cancer spheroids or biomaterials, embedded in a matrix (722a) is provided. Microfluidic molds/housing 612, 712 were prepared using standard photolithography techniques and devices were fabricated with polydimethylsiloxane (PDMS). The center (first) channel 620 contains fibroblast-embedded three-dimensional Matrigel into which biomaterial implants or three-dimensional spheroids is placed through a hole (port) 628, 728. The outer (second and third) channels 640, 660 (740, 760) serve to introduce macrophages and chemokines, respectively. Each channel is separated from adjacent channels by respective septa 630, 650 (730, 750) an array of micropillars 652 (visible in FIG. 6B, with corresponding micropillars 632 visible in FIGS. 6D, and 6E), 752 forming a plurality of openings 654 (634) therebetween. An indentation 629, 729 below the center channel 620, 720 enables the secure placement of biomaterials or spheroids within the device. The versatile device design allows for the introduction of multiple cell types, a combination of three-dimensional, two-dimensional, and fluidic culture, as well as introduction of various biomaterials, nanomaterials, cytokines/chemokines, therapeutic drugs, and other stimuli that can impact fibrosis formation. The device design can also be modified by adjusting the number of channels, channel heights, channel lengths, channel widths, indentation height, indentation shape, types of micropillars, sizes of micropillars, spacing between adjacent micropillars, and other aspects regarding the device feature dimensions. Using fluorescently-labeled molecules of different sizes, a cytokine gradient can be created throughout the device 610, enabling potential stimulation of cells with cytokines. Device 610 and similar devices can be used to study fibrosis of various target objects, for example, melanoma spheroids and alginate-based biomaterials.

### Example 2

L929 mouse fibroblasts and RAW264.7 mouse macrophages were fluorescently labelled with red and green CELLTRACKER® (Thermo Fisher Scientific) dyes respectively. Extensive studies were conducted to optimize cell seeding density, culture times, and biomaterial implantation. Cells were co-cultured two-dimensionally or within microfluidic devices described in Example 1, after which cancer spheroids or alginate-based biomaterials were implanted within the two-dimensional cultures and within the microfluidic devices. Immunostaining, fluorescence microscopy, Griess assays, and histological analysis were used to evaluate immune cell activation and fibrosis formation in response to the spheroids and implants. Griess Assays were performed on media collected from cultured cells. Co-culture of RAW264.7 macrophages with varying ratios of L929 fibroblasts led to similar levels of nitrite production as the culture of RAW264.7 macrophages alone, indicating that L929 cells do not activate RAW264.7 cells. In contrast, stimulating macrophages with lipopolysaccharide (LPS) led to macrophage activation, as indicated by nitrite production. Co-culture of RAW264.7 and L929 cells were visualized with CELLTRACKER® dyes. No morphological changes were observed as a result of the co-culture. F-actin visualization for RAW264.7 cells treated with LPS was performed. Morphological changes were observed with increasing concentrations of LPS. Co-culture of L929 and RAW264.7 cells was performed in the microfluidic device. L929 cells were cultured in 3D within the Matrigel channel, while macrophages were introduced in 2D from an adjacent channel. After 24 hours of co-culture, the cells were primarily retained within their respective channels.

In preliminary 2D experiments, it was found that L929 fibroblasts alone did not activate RAW264.7 macrophages and confirmed the effects of lipopolysaccharide (LPS) stimulation on macrophage morphology and activation. These measurements were then projected to the three-dimensional microfluidic system. The effects of cell count and incubation time on B16-F10 melanoma spheroid formation were also determined. That information was used to select optimal parameters to form spheroids for use in the microfluidic device. F-actin staining of fibroblasts was also performed.

Device characterization results and data from microfluidic co-cultures of fibroblasts and macrophages with melanoma spheroids and alginate-based biomaterials are projected.

### Example 3

Diffusion of fluorescently-labeled dextran molecules of different sizes from the lateral (side) channels into the central channel of the microfluidic device was observed during experiments. Experiments were run with 10 k molecular weight (MW) dextran. Experiments were also run with 10 k, 70 k MW dextran. Dextran diffusion was measured at zero hours, three hours, and twenty-four hours. Dextran molecules with molecular weights of 10 k and 70 k successfully diffused from a side channel (from the second or third channel toward the first (central) channel) across the Matrigel-filled channel of the device over a span of 24 hours. A linear gradient of dextran concentration (measured by fluorescence intensity) was observed across the device. These experiments confirm the potential for creation of a cytokine gradient within the device.

### Example 4

Macrophages and fibroblasts were co-cultured in a side channel and center channel, respectively, of the microfluidic device, and a suture was provided in the center channel during the co-culture to monitor any development of fibrosis on the suture. The suture can optionally be placed in the microfluidic device during assembly of the device.

### Example 5

Development of melanoma spheroids for placement in the microfluidic device was performed. Melanoma spheroid diameter was measured as a function of cell density and incubation time. Live-dead staining of melanoma spheroids formed with 8 cells/μL (i), 27 cells/μL (ii), and 54 cells/μL (iii) after 72 h of incubation was performed. Live cells and dead cells were labelled with distinguishing colors.

### Example 6

Placement of biomaterials in the microfluidic device was investigated. Alginate beads were implanted in the microfluidic device implant well (indentation/container). Alginate was chosen as a model biomaterial because it is known to lead to fibrosis. Placement of suture in the Fibrosis Chip. Matrigel successfully gelled around the suture. These results confirm the feasibility of biomaterial placement within the Fibrosis Chip. Suture placement is possible in devices even without the incorporation of an implant well.

### Example7

Inducing collagen deposition by fibroblasts in a two-dimensional model was studied. To determine whether L929 fibroblasts would deposit collagen in response to stimuli, well plate experiments were conducted following a protocol described in Good et al., “A High Content, Phenotypic ‘Scar-In-A-Jar’ Assay for Rapid Quantification of Collagen Fibrillogenesis Using Disease-Derived Pulmonary Fibroblasts,” BMC Biomedical Engineering, 1:14, 1-10 (2019), which is incorporated by reference herein in its entirety. Immunostaining experiments were carried out on L929 cells and primary mouse skin fibroblasts exposed to media containing L-ascorbic acid, FICOLL®, and TGF-β1. Some increases in Collagen I deposition and α-SMA expression by primary cells in response to TGF-β1 treatment were observed. In contrast, L929 cells had no α-SMA expression and did not have increased Collagen I deposition in response to TGF-β1 treatment. Additionally, the type of L-ascorbic acid used had some effects on Collagen I deposition. These findings indicate the relevance of and effects of cell type and L-ascorbic acid type on in vitro extracellular matrix deposition.

### Example 8

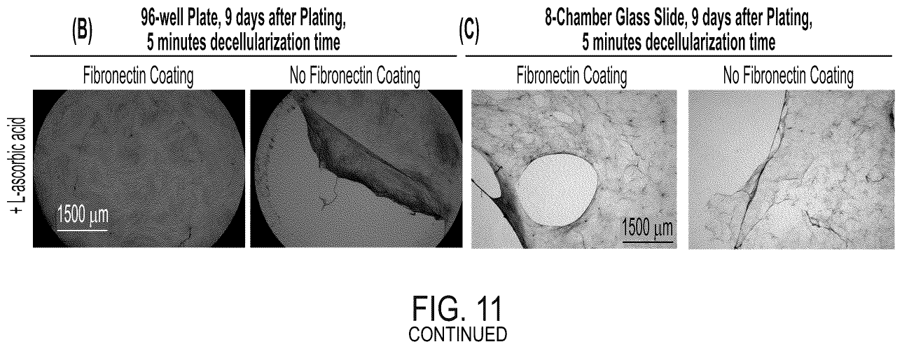

One method that can be used for visualization and quantification of collagen deposition in the microfluidic device is picrosirius red staining. Picrosirius red staining was performed on paraformaldehyde-fixed decellularized extracellular matrix deposited by WI-38 human lung cells on 2D surfaces revealed collagen deposition (FIG. 11). FIG. 11, part A, shows the results at one day and nine days after plating cells on Permanox plastic four chamber slides (250,000 cells/chamber). Cells were grown in chambers with or without a fibronectin coating and with or without L-asorbic acid. Cells grown in Permanox plastic 4-chamber slides (250,000 cells/chamber) exhibit an increase in collagen deposition with L-ascorbic acid treatment, as well as with longer culture times (nine days versus one day). In the absence of a fibronectin coating, no visible collagen deposition remained after 14-15 minutes of decellularization. FIG. 11, part B, shows the results of plating WI-38 cells grown in 96-well plates (44,444 cells/well) nine days after plating with a five minute decellularization time. Cells appear to deposit collagen fibers that are retained, even in the absence of a fibronectin coating, with a short decellularization time of five minutes. FIG. 11, part C, shows the results of plating WI-38 cells grown in eight chamber glass slides (97,222 cells/chamber) nine days after plating with a five minute decellularization time. Cells appear to deposit collagen fibers that are retained, even in the absence of a fibronectin coating, with a short decellularization time of five minutes.

### Example 9

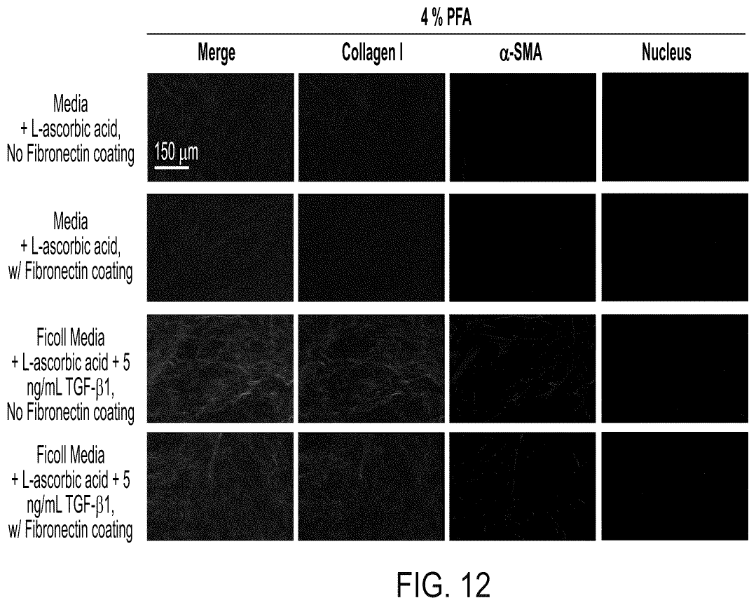

The use of different materials within the microfluidic device is of interest because it has been found that cells sometimes behave differently on plastic surfaces compared to glass surfaces (FIGS. 12-17). The material used for fabrication of the microfluidic device, in addition to any materials that are placed in the device, can play a role in how the cells will behave. The processing of the cells after culture (for example, fixation method and decellularization) can also affect the visualization of proteins of interest such as Collagen I and alpha-smooth muscle cell actin (α-SMA) (FIGS. 12-17). A variety of different processing and staining methods can be used with the microfluidic device.

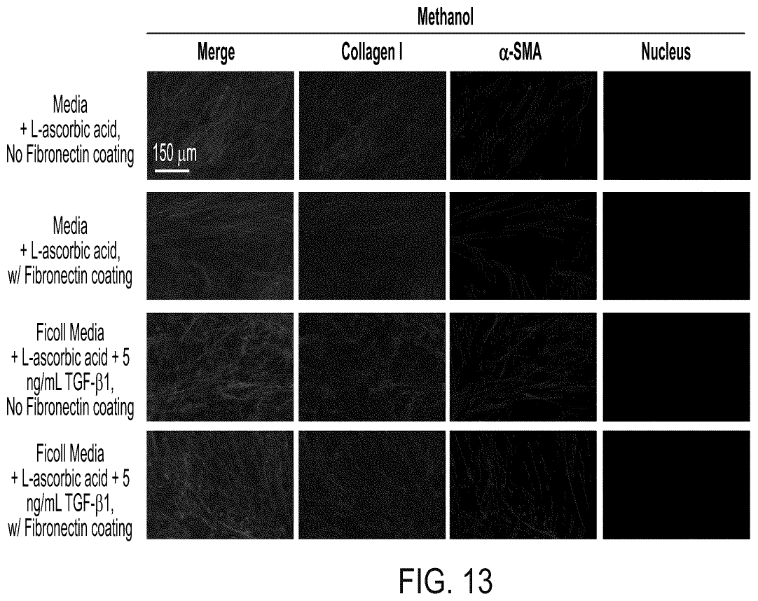

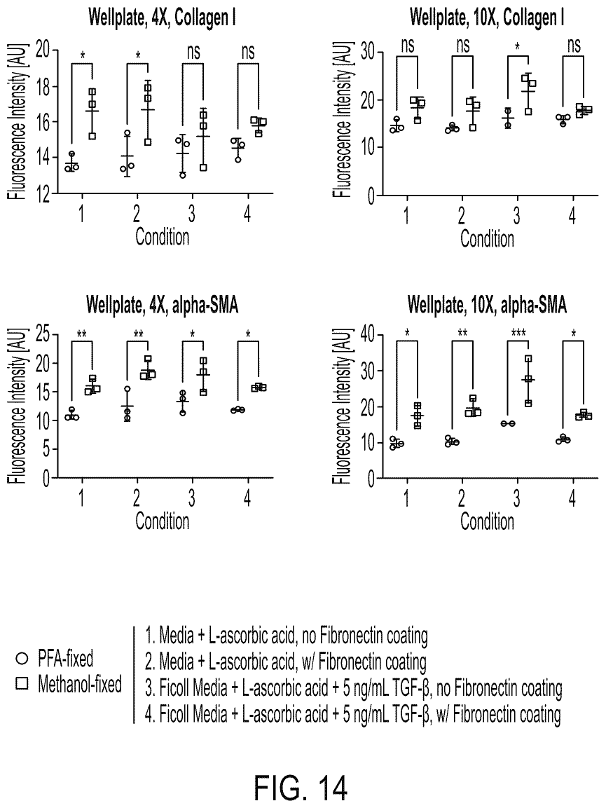

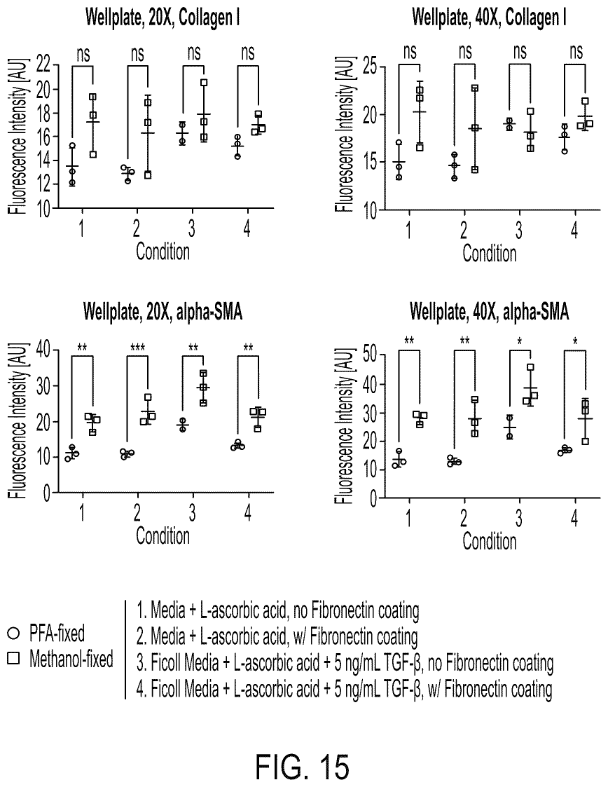

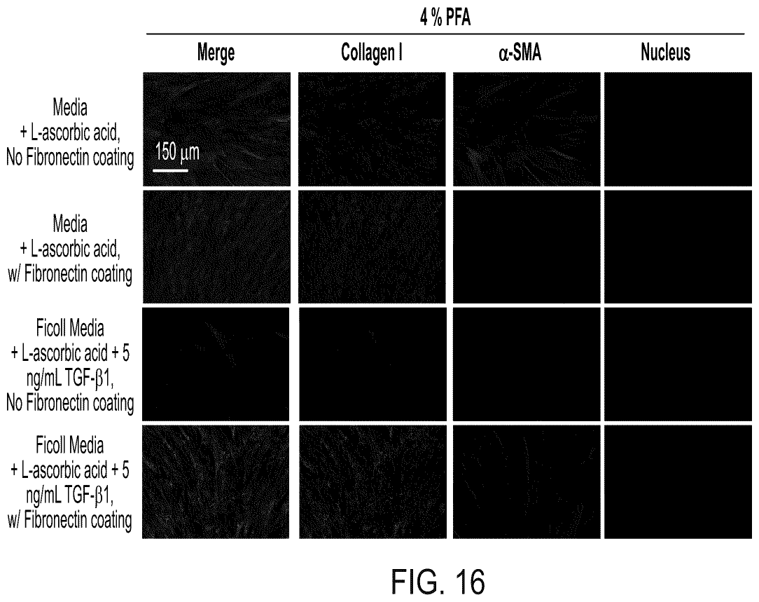

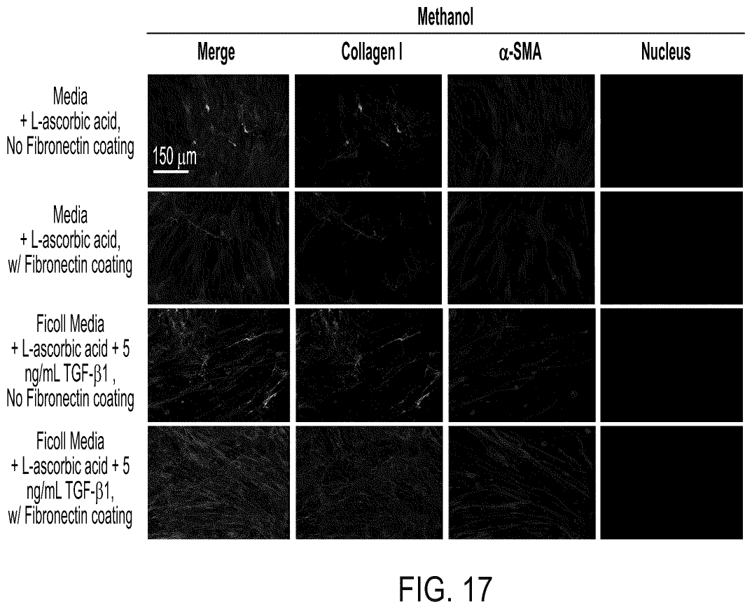

Collagen I and α-SMA deposition by WI-38 cells in 96-well plates was studied. Cells were plated at 44,444 cells/well and grown on media including L-asorbic acid. Cells were grown in wells that either had no coating or a fibronectin coating. Cells were grown with or without 5 ng/ml TGF-β1. Cells were fixed with either 4% paraformaldehyde (PFA) (FIG. 12) or methanol (FIG. 13). Fluorescence images of “merge,” collagen I, α-SMA, and nucleus are shown in consecutive columns. Images shown are at 20× magnification. FIGS. 14 and 15 show quantified fluorescence intensity for collagen I and α-SMA channels. Image quantification was conducted in ImageJ, with mean image intensity calculated for each channel. Statistical analysis was conducted in GraphPad Prism using 2-way ANOVAs with multiple comparisons. Significant differences between Collagen I intensity in PFA-fixed versus methanol-fixed wells are apparent at low magnifications, but not at higher magnifications. In contrast, significant differences between α-SMA intensity in PFA-fixed versus methanol-fixed wells re apparent at all tested magnifications. FIGS. 16 and 17 show the results of cell studies analogous to those shown in FIGS. 12 and 13 but with the cells grown in eight chamber glass slides. Cells were plated at a concentration of 97,222 cells/chamber for regular media treatments and 18,421 cells/chamber for Ficoll Media treatments. Here again, an improvement in staining is observed in methanol-fixed cells compared to PFA-fixed cells. Additionally, Collagen I appears to be fibrillar in methanol-fixed cells, but appears to localize around the nucleus in the PFA-fixed cells.

The present disclosure can include any combination of these various features or embodiments above and/or below as set forth in sentences and/or paragraphs. Any combination of disclosed features herein is considered part of the present disclosure.
1. A fibrotic diagnostic system comprising:
   a microfluidic device comprising:
      a device housing comprising a device interior;
      a plurality of channels within the device interior, the plurality of channels comprising:
         a first channel configured to receive a target object, a matrix, and a first biological composition configured to promote fibrosis of the target object,
         a second channel configured to receive a second biological composition configured to promote fibrosis of the target object,
         a first septum separating the first and second channels and comprising a first plurality of openings providing fluid communication between the first and second channels,
         a third channel configured to receive a third biological composition configured to promote fibrosis of the target object, and
         a second septum separating the first and third channels and comprising a second plurality of openings providing fluid communication between the first and third channels; and
      a plurality of a channel ports in the device housing in fluid communication with the plurality of channels, the plurality of channel ports comprising a first set of channel ports in fluid communication with the first channel, a second set of channel ports in fluid communication with the second channel, and a third set of channel ports in fluid communication the third channel, wherein each set of channel ports comprises one or more channel ports.
2. The fibrotic diagnostic system of any preceding or following embodiment/feature/aspect wherein the system is configured to form a gradient of at least one type of molecule across the first channel, the second channel, or the third channel, or any combination thereof.
3. The fibrotic diagnostic system of any preceding or following embodiment/feature/aspect wherein the at least one type of molecule comprises a chemotactic molecule.
4. The fibrotic diagnostic system of any preceding or following embodiment/feature/aspect wherein the chemotactic molecule promotes the movement of at least one cell type present in the first biological composition, the second biological composition, or the third biological composition, or any combination thereof.
5. The fibrotic diagnostic system of any preceding or following embodiment/feature/aspect wherein the first biological composition, the second biological composition, or the third biological composition, or any combination thereof comprises the at least one type of molecule.
6. The fibrotic diagnostic system of any preceding or following embodiment/feature/aspect, wherein the second and third sets of channel ports each comprise two or more channel ports.
7. The fibrotic diagnostic system of any preceding or following embodiment/feature/aspect, wherein each set of channel ports comprises two or more channel ports.
8. The fibrotic diagnostic system of any preceding or following embodiment/feature/aspect, wherein the second and third sets of channel ports each comprise a first channel port and a second channel port, the first channel port proximal a first end of its respective channel and the second channel proximal a second end of its respective channel.
9. The fibrotic diagnostic system of any preceding or following embodiment/feature/aspect, wherein each of the sets of channel ports comprises a first channel port and a second channel port, the first channel port proximal a first end of its respective channel and the second channel proximal a second end of its respective channel.
10. The fibrotic diagnostic system of any preceding or following embodiment/feature/aspect, wherein the first channel comprises a central channel port configured to receive the target object into the first channel.
11. The fibrotic diagnostic system of any preceding or following embodiment/feature/aspect, wherein the central channel port is between a first end of the first channel and a second end of the second channel.
12. The fibrotic diagnostic system of any preceding or following embodiment/feature/aspect, wherein one or more of the plurality of channel ports comprises a seal, valve, or closure, or any combination thereof configured for movement between an open state and a closed state.
13. The fibrotic diagnostic system of any preceding or following embodiment/feature/aspect comprising a retention element comprised by one or more of the first, second, and third channels, the retention element configured to retain at least one component insertable into one or more of the first, second, and third channels.
14. The fibrotic diagnostic system of any preceding or following embodiment/feature/aspect, wherein the retention element comprises a modification of a channel surface.
15. The fibrotic diagnostic system of any preceding or following embodiment/feature/aspect, wherein the modification comprises a biomolecule.
16. The fibrotic diagnostic system of any preceding or following embodiment/feature/aspect, wherein the biomolecule comprises a polypeptide, a polysaccharide, a lipid, or a polynucleotide, or any combination thereof.
17. The fibrotic diagnostic system of any preceding or following embodiment/feature/aspect, wherein the modification comprises a polymer, a metal, or a ceramic, or any combination thereof.
18. The fibrotic diagnostic system of any preceding or following embodiment/feature/aspect, wherein the device housing comprises a polymer, a metal, or a ceramic, or any combination thereof.
19. The fibrotic diagnostic system of any preceding or following embodiment/feature/aspect, wherein the device housing comprises a first polymer and the modification comprises a second polymer, the first and second polymers differing from each other in chemical composition.
20. The fibrotic diagnostic system of any preceding or following embodiment/feature/aspect, wherein the modification comprises a physical structure.
21. The fibrotic diagnostic system of any preceding or following embodiment/feature/aspect, wherein the physical structure and the device housing comprise a common material.
22. The fibrotic diagnostic system of any preceding or following embodiment/feature/aspect, wherein the modification comprises a container for receiving the target object.
23. The fibrotic diagnostic system of any preceding or following embodiment/feature/aspect, wherein the container comprises a recess in the channel surface.
24. The fibrotic diagnostic system of any preceding or following embodiment/feature/aspect, wherein the physical structure comprises a plurality of physical structures.
25. The fibrotic diagnostic system of any preceding or following embodiment/feature/aspect, wherein the plurality of physical structures comprises a plurality of recesses in the channel surface, or a plurality of protrusions from the channel surface, or both.
26. The fibrotic diagnostic system of any preceding or following embodiment/feature/aspect, wherein the first channel, the second channel, or the third channel, or any combination thereof comprises a curvilinear cross-section, or a rectilinear cross-section, or both.
27. The fibrotic diagnostic system of any preceding or following embodiment/feature/aspect, wherein the first channel, the second channel, or the third channel, or any combination thereof comprises a constant width, or a constant diameter, or both.
28. The fibrotic diagnostic system of any preceding or following embodiment/feature/aspect, wherein the first channel, the second channel, or the third channel, or any combination thereof comprises a variable width, or a constant diameter, or both.
29. The fibrotic diagnostic system of any preceding or following embodiment/feature/aspect, wherein two or more channels of the plurality of channels have a common length.
30. The fibrotic diagnostic system of any preceding or following embodiment/feature/aspect, wherein two or more channels of the plurality of channels are parallel to one another.
31. The fibrotic diagnostic system of any preceding or following embodiment/feature/aspect, wherein the first channel is between the second and third channels.
32. The fibrotic diagnostic system of any preceding or following embodiment/feature/aspect, wherein two or more of the first, second, and third channels differ from another with respect to a volume, a surface area, a cross-sectional area, length, a width, or a diameter, or any combination thereof.
33. The fibrotic diagnostic system of any preceding or following embodiment/feature/aspect, wherein a width, a diameter, or both of the first channel is greater than a width, a diameter, or both of the second channel, or of the third channel, or of both.
34. The fibrotic diagnostic system of any preceding or following embodiment/feature/aspect, wherein a cross-sectional area, a width, a diameter, or any combination thereof of one or more of the first, second, and third channels differs along its length.
35. The fibrotic diagnostic system of any preceding or following embodiment/feature/aspect, wherein one or more of the first, second, and third channels comprises a first peripheral segment proximate a first end of the channel, a second peripheral segment proximate a second end of the channel, and a central segment between the first and second peripheral segments.
36. The fibrotic diagnostic system of any preceding or following embodiment/feature/aspect, wherein the central segment comprises a cross-sectional area, a width, or a diameter, or any combination thereof that is greater than a cross-section area, a width, or a diameter, or any combination thereof of one or both of the first and second peripheral segments.
37. The fibrotic diagnostic system of any preceding or following embodiment/feature/aspect, wherein the central segment of the first channel comprises a cross-sectional area, a width, or a diameter, or any combination thereof that is greater than a cross-sectional area, a width, or a diameter, or any combination thereof of one or both of the first and second peripheral segments of the first channel.
38. The fibrotic diagnostic system of any preceding or following embodiment/feature/aspect, wherein one or both of the first and second peripheral segments are colinear with the central segment in relation to their respective central longitudinal axes.
39. The fibrotic diagnostic system of any preceding or following embodiment/feature/aspect, wherein one or both of the first and second peripheral segments form an angle other than 180° with the central segment in relation to their respective central longitudinal axes.
40. The fibrotic diagnostic system of any preceding or following embodiment/feature/aspect, wherein the angle comprises a right angle, an acute angle, or an obtuse angle, or any combination thereof.
41. The fibrotic diagnostic system of any preceding or following embodiment/feature/aspect, wherein two or more channels of the plurality of channels lie in a common plane.
42. The fibrotic diagnostic system of any preceding or following embodiment/feature/aspect, wherein the first, second, and third channels of the plurality of channels lie in a common plane.
43. The fibrotic diagnostic system of any preceding or following embodiment/feature/aspect, wherein a length of the first septum is less than a length of the first channel, or a length of the second channel, or both.
44. The fibrotic diagnostic system of any preceding or following embodiment/feature/aspect, wherein a length of the second septum is less than a length of the first channel, or a length of the third channel, or both.
45. The fibrotic diagnostic system of any preceding or following embodiment/feature/aspect, wherein the first septum, the second septum, or both comprises a membrane.
46. The fibrotic diagnostic system of any preceding or following embodiment/feature/aspect, wherein the membrane comprises a porous membrane.
47. The fibrotic diagnostic system of any preceding or following embodiment/feature/aspect, wherein the membrane comprises a semipermeable membrane.
48. The fibrotic diagnostic system of any preceding or following embodiment/feature/aspect, wherein the membrane comprises a biological membrane, or a synthetic membrane, or both.
49. The fibrotic diagnostic system of any preceding or following embodiment/feature/aspect, wherein the first septum comprises a first plurality of columns separated by the first plurality of openings, the second septum comprises a second plurality of columns separated by the second plurality of second openings, or both.
50. The fibrotic diagnostic system of any preceding or following embodiment/feature/aspect, wherein the first plurality of columns, or the second plurality of columns, or both have a rectilinear cross-section, a curvilinear cross-section, or both.
51. The fibrotic diagnostic system of any preceding or following embodiment/feature/aspect, wherein the first plurality of columns, or the second plurality of columns, or both are trapezoidal.
52. The fibrotic diagnostic system of any preceding or following embodiment/feature/aspect, wherein:
   the openings of the first plurality of openings differ in number, size, shape, or spacing, or any combination thereof; or
   the openings of the second plurality of openings differs in number, size, shape, or spacing, or any combination thereof; or both.
53. The fibrotic diagnostic system of any preceding or following embodiment/feature/aspect, wherein:
   the openings of the first plurality of openings differ in number, size, shape, or spacing, or any combination thereof along one or more dimension of the first septum; or
   the openings of the second plurality of openings differs in number, size, shape, or spacing, or any combination thereof along one or more dimension of the second septum; or both.
54. The fibrotic diagnostic system of any preceding or following embodiment/feature/aspect, wherein the first plurality of openings and the second plurality of openings differ from one another.
55. The fibrotic diagnostic system of any preceding or following embodiment/feature/aspect, wherein the device housing comprises a substrate and the plurality of channels and the plurality of channel ports are formed in the substrate.
56. The fibrotic diagnostic system of any preceding or following embodiment/feature/aspect, wherein the device housing comprises a silicone.
57. The fibrotic diagnostic system of any preceding or following embodiment/feature/aspect, wherein the device housing comprises polydimethylsiloxane (PDMS).
58. The fibrotic diagnostic system of any preceding or following embodiment/feature/aspect, wherein the device housing comprises a flexible material, or a rigid material, or both.
59. The fibrotic diagnostic system of any preceding or following embodiment/feature/aspect, wherein the device housing comprises a conductor, or an insulator, or both.
60. The fibrotic diagnostic system of any preceding or following embodiment/feature/aspect, wherein the substrate comprises a first material and one or more channel surfaces comprise a second material differing from the first material.
61. The fibrotic diagnostic system of any preceding or following embodiment/feature/aspect, wherein the second material is distinct from the contents of one or more channels in which it is present.
62. The fibrotic diagnostic system of any preceding or following embodiment/feature/aspect, wherein the second material is a coating on the first material.
63. The fibrotic diagnostic system of any preceding or following embodiment/feature/aspect, wherein one or more channel surfaces are coated with a plurality of different materials.
64. The fibrotic diagnostic system of any preceding or following embodiment/feature/aspect, wherein one or more channel surfaces are coated with a plurality of coatings.
65. The fibrotic diagnostic system of any preceding or following embodiment/feature/aspect, wherein at least two of the plurality of coatings differ in material composition.
66. The fibrotic diagnostic system of any preceding or following embodiment/feature/aspect, wherein the substrate comprises a first material, and the plurality of coatings comprises a first coating comprising a second material adjacent the substrate on one or more channel surfaces and a second coating comprising a third material overlaying the first coating.
67. The fibrotic diagnostic system of any preceding or following embodiment/feature/aspect, wherein the first and second materials differ from one another and the second and third materials differ from one another.
68. The fibrotic diagnostic system of any preceding or following embodiment/feature/aspect, wherein the first, second, and third materials differ from one another.
69. The fibrotic diagnostic system of any preceding or following embodiment/feature/aspect, wherein the plurality of coatings comprise at least two layers differing in average thickness from one another.
70. The fibrotic diagnostic system of any preceding or following embodiment/feature/aspect, wherein a material composition, a thickness, or patterning, or a combination thereof of a coating on one or more channel surfaces differ along the lengths of one or more channels.
71. The fibrotic diagnostic system of any preceding or following embodiment/feature/aspect, wherein two or more of the channels differ in material composition of their surface coatings.
72. The fibrotic diagnostic system of any preceding or following embodiment/feature/aspect, wherein the first channel comprises a first coating, the second channel comprises a second coating, and the third channel comprises a third coating; the first, second, and third coating differing in a material composition, a thickness, or patterning, or a combination thereof.
73. The fibrotic diagnostic system of any preceding or following embodiment/feature/aspect, wherein the first, second, and third channels are coated with the same material.
74. The fibrotic diagnostic system of any preceding or following embodiment/feature/aspect, wherein one or more coatings comprise an organic material, or an inorganic material, or both.
75. The fibrotic diagnostic system of any preceding or following embodiment/feature/aspect, wherein one or more coatings comprise a polymer.
76. The fibrotic diagnostic system of any preceding or following embodiment/feature/aspect, wherein one or more coatings comprise a biomolecule.
77. The fibrotic diagnostic system of any preceding or following embodiment/feature/aspect, wherein one or more coatings comprise a protein, or a nucleic acid, a lipid, or a carbohydrate, or any combination thereof.
78. The fibrotic diagnostic system of any preceding or following embodiment/feature/aspect, wherein one or more coatings comprise a fibronectin, an extracellular matrix-derived composition, a basement membrane extract, a decellularized extracellular matrix, a solubilized basement membrane preparation, a mixture of extracellular matrix proteins, a tumor derived composition, Matrigel, or a hyaluronic acid, or any combination thereof.
79. The fibrotic diagnostic system of any preceding or following embodiment/feature/aspect, wherein the substrate comprises silicon and one or more channel surfaces are coated with a coating comprises a silane.
80. The fibrotic diagnostic system of any preceding or following embodiment/feature/aspect, wherein one or more channel surfaces are subjected to silanization.
81. The fibrotic diagnostic system of any preceding or following embodiment/feature/aspect, wherein one or more channel surfaces are treated with plasma.
82. The fibrotic diagnostic system of any preceding or following embodiment/feature/aspect, wherein the plasma comprises oxygen.
83. The fibrotic diagnostic system of any preceding or following embodiment/feature/aspect, wherein one or more coatings are applied by emersion, spraying, brushing, injection, precipitation, polymerization, or vapor deposition, or any combination thereof.
84. The fibrotic diagnostic system of any preceding or following embodiment/feature/aspect, wherein one or more channel surfaces comprise a laminate.
85. The fibrotic diagnostic system of any preceding or following embodiment/feature/aspect, wherein the device housing comprises a transparent material, a translucent material, a tinted material, a colored material, a polarizing material, a material that selectively transmits wavelengths of electromagnetic radiation, a material that selectively refracts wavelengths of electromagnetic radiation, a material that selectively reflects wavelengths of electromagnetic radiation, or an opaque material, or any combination thereof.
86. The fibrotic diagnostic system of any preceding or following embodiment/feature/aspect, wherein the device housing comprises a window permitting a view of the device interior.
87. The fibrotic diagnostic system of any preceding or following embodiment/feature/aspect, wherein the view is of one or more channel of the plurality of channels.
88. The fibrotic diagnostic system of any preceding or following embodiment/feature/aspect, wherein the view comprises a view of the first channel, or the target object, or both.
89. The fibrotic diagnostic system of any preceding or following embodiment/feature/aspect, wherein the device housing comprises a plurality of windows permitting a plurality of views of the device interior.
90. The fibrotic diagnostic system of any preceding or following embodiment/feature/aspect, wherein the target object comprises an organic material, or an inorganic material, or both.
91. The fibrotic diagnostic system of any preceding or following embodiment/feature/aspect, wherein the target object comprises a tissue, or a medical device, or both.
92. The fibrotic diagnostic system of any preceding or following embodiment/feature/aspect, wherein the target object comprises a spheroid, an organoid, an alginate-based biomaterial, a scaffold-supported cellular structure, or a bio-printed structure, or any combination thereof.
93. The fibrotic diagnostic system of any preceding or following embodiment/feature/aspect, wherein the target object comprises a neoplastic cell, a malignant cell, or a benign cell, or any combination thereof.
94. The fibrotic diagnostic system of any preceding or following embodiment/feature/aspect, wherein the target comprises one or more coatings.
95. The fibrotic diagnostic system of any preceding or following embodiment/feature/aspect, wherein the one or more coatings of the target are distinct from the matrix.
96. The fibrotic diagnostic system of any preceding or following embodiment/feature/aspect, wherein the one or more coatings of the target differ from one or more coatings of one or more channel surfaces with respect to a material composition, a thickness, or patterning, or a combination thereof.
97. The fibrotic diagnostic system of any preceding or following embodiment/feature/aspect, wherein the one or more coatings of the target are the same as one or more coatings of one or more channel surfaces with respect to a material composition, a thickness, or patterning, or a combination thereof.
98. The fibrotic diagnostic system of any preceding or following embodiment/feature/aspect, wherein the target is coated with a plurality of different materials.
99. The fibrotic diagnostic system of any preceding or following embodiment/feature/aspect, wherein the target is coated with a plurality of coatings.
100. The fibrotic diagnostic system of any preceding or following embodiment/feature/aspect, wherein at least two of the plurality of coatings differ in material composition, thickness, or patterning, or a combination thereof.
101. The fibrotic diagnostic system of any preceding or following embodiment/feature/aspect, wherein the plurality of coatings comprises a first coating comprising a first material adjacent the target and a second coating comprising a second material overlaying the first coating.
102. The fibrotic diagnostic system of any preceding or following embodiment/feature/aspect, wherein the target is coated prior to placement in the microfluidic device.
103. The fibrotic diagnostic system of any preceding or following embodiment/feature/aspect, wherein the matrix comprises a hydrogel.
104. The fibrotic diagnostic system of any preceding or following embodiment/feature/aspect, wherein the matrix comprises a fibronectin, an extracellular matrix-derived composition, a basement membrane extract, a decellularized extracellular matrix, a solubilized basement membrane preparation, a mixture of extracellular matrix proteins, a tumor derived composition, Matrigel, or a hyaluronic acid, or any combination thereof.
105. The fibrotic diagnostic system of any preceding or following embodiment/feature/aspect, wherein the matrix comprises a natural polymer, an artificial polymer, or a cross-linked polymer, or any combination thereof.
106. The fibrotic diagnostic system of any preceding or following embodiment/feature/aspect, wherein the matrix comprises a polysaccharide, a polypeptide, a lipid, a polynucleotide, a hyaluronic acid, a hyaluronic acid methacrylate, a hyaluronic acid diacrylate, a fibronectin, a fibrinogen, a collagen, or a methacrylate collagen, or any combination thereof.
107. The fibrotic diagnostic system of any preceding or following embodiment/feature/aspect, wherein the first biological composition comprises a fibroblast, or an endothelial cell, or both.
108. The fibrotic diagnostic system of any preceding or following embodiment/feature/aspect, wherein the first biological composition comprises a blood serum, an ascorbic acid, a retinoic acid, proline, or bleomycin, or any combination thereof.
109. The fibrotic diagnostic system of any preceding or following embodiment/feature/aspect, wherein the first biological composition comprises a biomolecule promoting fibrosis, a complement protein, a blood clotting associated protein, a bacterial cell wall protein, or a lipopolysaccharide (LPS), or any combination thereof.
110. The fibrotic diagnostic system of any preceding or following embodiment/feature/aspect, wherein the second biological composition comprises at least one type of biological cell capable of promoting fibrosis.
111. The fibrotic diagnostic system of any preceding or following embodiment/feature/aspect, wherein the second biological composition comprises a factor that promotes differentiation of a biological cell into a type of biological cell capable of promoting fibrosis, a factor that promotes expression of a biomolecule that promotes fibrosis, or a factor that promotes expansion of a type of biological cell capable of promoting fibrosis, or any combination thereof.
112. The fibrotic diagnostic system of any preceding or following embodiment/feature/aspect, wherein the second biological composition comprises blood serum, a platelet, a white blood cell, a macrophage, a monocyte, a peripheral blood mononuclear cell (PBMC), a stem cell, a mast cell, a dendritic cell, a neutrophil, a granulocyte, a T cell, or a B cell, or any combination thereof.
113. The fibrotic diagnostic system of any preceding or following embodiment/feature/aspect, wherein the third biological composition comprises a population of molecules capable of forming a gradient radiating from the third channel into the first channel, or into both the first and second channels.
114. The fibrotic diagnostic system of any preceding or following embodiment/feature/aspect, wherein the third biological composition comprises a cytokine, a chemokine, a cell nutrient, a transcription factor, or a growth factor, or any combination thereof.
115. The fibrotic diagnostic system of any preceding or following embodiment/feature/aspect, wherein the third biological composition comprises CCL-2/MCP-1, CCL3, CCL11, fibroblast growth factor (FGF), an interferon, IFN-gamma, an interleukin (IL), IL-4, IL-1α, IL-1β, IL-2, IL-5, IL-6, IL-7, IL-9, IL-13, IL-17A, IL-17F, IL-21,IL-22, IL-25, platelet derived growth factor (PDGF), TGF-alpha, TGF-beta, granulocyte colony-stimulating factor (G-CSF), macrophage colony-stimulating factor (M-CSF), granulocyte macrophage colony-stimulating factor (GM-CSF), prothrombin, or thrombin, or any combination thereof.
116. The fibrotic diagnostic system of any preceding or following embodiment/feature/aspect, wherein the at least one type of molecule of the gradient comprises a cytokine, a chemokine, a cell nutrient, a transcription factor, or a growth factor, or any combination thereof.
117. The fibrotic diagnostic system of any preceding or following embodiment/feature/aspect, wherein the at least one type of molecule of the gradient comprises a transforming growth factor beta (TGF-beta).
118. The fibrotic diagnostic system of any preceding or following embodiment/feature/aspect, wherein the TGF-beta comprises TGF-beta1.
119. The fibrotic diagnostic system of any preceding or following embodiment/feature/aspect, wherein the first biological composition, the second biological composition, or the third biological composition, or any combination thereof comprises a labeled cell, or a labeled biomolecule, or both.
120. The fibrotic diagnostic system of any preceding or following embodiment/feature/aspect, wherein the first biological composition, the second biological composition, or the third biological composition, or any combination thereof comprises a compound having known or potential anti-fibrotic efficacy.
121. The fibrotic diagnostic system of any preceding or following embodiment/feature/aspect further comprising the matrix, the first biological composition, the second biological composition, or the third biological composition, or any combination thereof.
122. The fibrotic diagnostic system of any preceding or following embodiment/feature/aspect further comprising the target object.
123. The fibrotic diagnostic system of any preceding or following embodiment/feature/aspect further comprising a gradient of at least one molecule across the first channel, the second channel, or the third channel, or any combination thereof.
124. The fibrotic diagnostic system of any preceding or following embodiment/feature/aspect, wherein one or more components of the matrix, the first biological composition, the second biological composition, or the third biological composition, or any combination thereof is anchored to a channel surface.
125. The fibrotic diagnostic system of any preceding or following embodiment/feature/aspect, further comprising a detector, the detector comprising a sensor configured to detect fibrosis or characteristic indicative of the same of the target object.
126. The fibrotic diagnostic system of any preceding or following embodiment/feature/aspect, wherein the detector is configured to receive the microfluidic device.
127. The fibrotic diagnostic system of any preceding or following embodiment/feature/aspect, wherein the detector comprises a detector housing comprising a detector interior.
128. The fibrotic diagnostic system of any preceding or following embodiment/feature/aspect, wherein the detector further comprises a receptacle configured to receive the microfluidic device, the receptacle comprising an opening in the detector housing.
129. The fibrotic diagnostic system of any preceding or following embodiment/feature/aspect further comprising a thermal subsystem.
130. The fibrotic diagnostic system of any preceding or following embodiment/feature/aspect where the thermal subsystem comprises a heater, or a cooler, or both.
131. The fibrotic diagnostic system of any preceding or following embodiment/feature/aspect wherein the thermal subsystem is configured to regulate a temperature of the microfluidic device.
132. The fibrotic diagnostic system of any preceding or following embodiment/feature/aspect wherein the detector comprises the thermal subsystem.
133. The fibrotic diagnostic system of any preceding or following embodiment/feature/aspect wherein the thermal subsystem is configured to regulate the temperature of the microfluidic device when it is in the receptacle.
134. The fibrotic diagnostic system of any preceding or following embodiment/feature/aspect further comprising a fluidic subsystem configured for fluidic communication with the microfluidic device.
135. The fibrotic diagnostic system of any preceding or following embodiment/feature/aspect, wherein the fluidic subsystem is configured to load one or more component into the microfluidic device.
136. The fibrotic diagnostic system of any preceding or following embodiment/feature/aspect, wherein the fluidic subsystem is configured to supply a solid, a liquid, or a gas, or any combination thereof to the microfluidic device.
137. The fibrotic diagnostic system of any preceding or following embodiment/feature/aspect, wherein the fluidic subsystem is configured to load the target object, the matrix, the first biological composition, the second biological composition, or the third biological composition, or any combination thereof into the microfluidic device.
138. The fibrotic diagnostic system of any preceding or following embodiment/feature/aspect, wherein the fluidic subsystem is configured to supply one or more nutrients to the microfluidic device.
139. The fibrotic diagnostic system of any preceding or following embodiment/feature/aspect, wherein the fluidic subsystem is configured to regulate a pressure in an interior of the microfluidic device.
140. The fibrotic diagnostic system of any preceding or following embodiment/feature/aspect, wherein the fluidic subsystem is configured to regulate a gradient across the first channel, the second channel, or the third channel, or any combination thereof.
141. The fibrotic diagnostic system of any preceding or following embodiment/feature/aspect, wherein the fluidic subsystem is configured to remove one or more component from the microfluidic device.
142. The fibrotic diagnostic system of any preceding or following embodiment/feature/aspect wherein the detector comprises the fluidic subsystem.
143. The fibrotic diagnostic system of any preceding or following embodiment/feature/aspect wherein the fluidic subsystem is configured for fluidic communication with the microfluidic device when the microfluidic device is in the receptacle.
144. The fibrotic diagnostic system of any preceding or following embodiment/feature/aspect, wherein the sensor is configured to detect a particle, or a wave, or both emitted from the target object, or the microfluidic device, or both indicative of fibrosis.
145. The fibrotic diagnostic system of any preceding or following embodiment/feature/aspect, wherein the sensor is configured to detect electromagnetic radiation.
146. The fibrotic diagnostic system of any preceding or following embodiment/feature/aspect, wherein the electromagnetic radiation comprises ultraviolet radiation, visible light, or infrared radiation, or any combination thereof.
147. The fibrotic diagnostic system of any preceding or following embodiment/feature/aspect, wherein the electromagnetic radiation comprises fluorescence, or phosphorescence, or both.
148. The fibrotic diagnostic system of any preceding or following embodiment/feature/aspect, wherein the detector further comprises a particle source, or a wave source, or both configured to transmit a particle, a wave, or both to the target object, or the microfluidic device, or both.
149. The fibrotic diagnostic system of any preceding or following embodiment/feature/aspect, further comprising a source of electromagnetic radiation.
150. The fibrotic diagnostic system of any preceding or following embodiment/feature/aspect, wherein the detector comprises the source of electromagnetic radiation.
151. The fibrotic diagnostic system of any preceding or following embodiment/feature/aspect, wherein the source of electromagnetic radiation comprises an light emitting diode, a laser, an incandescent light source, an arc light source, a halogen light source, a chemical vapor light source, or a fluorescent light source, or any combination thereof.
152. The fibrotic diagnostic system of any preceding or following embodiment/feature/aspect, wherein the detector further comprises an optical subsystem configured to pass radiation, or affect radiation, or both.
153. The fibrotic diagnostic system of any preceding or following embodiment/feature/aspect, wherein the optical subsystem comprises one or more lenses, one or more filters, one or more mirrors, or one or more diffraction gratings, or any combination thereof.
154. The fibrotic diagnostic system of any preceding or following embodiment/feature/aspect, wherein the optical subsystem, or the sensor, or both comprise a microscope or one or more components thereof.
155. The fibrotic diagnostic system of any preceding or following embodiment/feature/aspect, there the optical subsystem comprises the sensor, or the source of electromagnetic radiation, or both
156. The fibrotic diagnostic system of any preceding or following embodiment/feature/aspect, wherein the sensor is configured to generate an output signal based on an input signal detected by the sensor.
157. The fibrotic diagnostic system of any preceding or following embodiment/feature/aspect, wherein the detector further comprises a transmitter, a receiver, a transceiver, or any combination thereof configured to receive information, or send information, or both.
158. The fibrotic diagnostic system of any preceding or following embodiment/feature/aspect, wherein the information comprises the output signal.
159. The fibrotic diagnostic system of any preceding or following embodiment/feature/aspect, wherein the information comprises an operational signal for operation of the sensor.
160. The fibrotic diagnostic system of any preceding or following embodiment/feature/aspect, wherein the detector further comprises a data port.
161. The fibrotic diagnostic system of any preceding or following embodiment/feature/aspect, further comprising a processor configured to receive and process information.
162. The fibrotic diagnostic system of any preceding or following embodiment/feature/aspect, wherein the processor is configured to control one or more subsystems or components of the fibrotic diagnostic system.
163. The fibrotic diagnostic system of any preceding or following embodiment/feature/aspect, wherein the processor is configured to control the sensor, the source of electromagnetic radiation, the optical subsystem, the thermal subsystem, or the fluidic subsystem, or any combination thereof.
164. The fibrotic diagnostic system of any preceding or following embodiment/feature/aspect, wherein the processor is configured to receive the output the signal from the sensor, to receive an operational signal from a user input, or both.
165. The fibrotic diagnostic system of any preceding or following embodiment/feature/aspect, wherein the detector comprises the processor.
166. The fibrotic diagnostic system of any preceding or following embodiment/feature/aspect, further comprises a memory configured for communication with the processor.
167. The fibrotic diagnostic system of any preceding or following embodiment/feature/aspect, wherein the memory is configured to store a program that controls the fibrotic diagnostic system or a component thereof.
168. The fibrotic diagnostic system of any preceding or following embodiment/feature/aspect, wherein the processor comprises the memory.
169. The fibrotic diagnostic system of any preceding or following embodiment/feature/aspect, wherein the detector comprises the memory.
170. The fibrotic diagnostic system of any preceding or following embodiment/feature/aspect, further comprising a user interface.
171. The fibrotic diagnostic system of any preceding or following embodiment/feature/aspect, wherein the user interface comprises a monitor, a button, a keyboard, a mouse, a light signal, a camera, an eye piece lens, a speaker, or a microphone, or any combination thereof.
172. The fibrotic diagnostic system of any preceding or following embodiment/feature/aspect, wherein the detector comprises the user interface.
173. The fibrotic diagnostic system of any preceding or following embodiment/feature/aspect, further comprising a power source, or a power interface, or both.
174. The fibrotic diagnostic system of any preceding or following embodiment/feature/aspect, wherein the detector comprises the power source, or the power interface, or both.
175. The fibrotic diagnostic system of any preceding or following embodiment/feature/aspect, further comprising a kit, the kit comprising the microfluidic device and one or more reagents for use in the microfluidic device.
176. The fibrotic diagnostic system of any preceding or following embodiment/feature/aspect, further comprising a kit, the kit comprising:
   the microfluidic device; and
   the matrix, the first biological composition, the second biological composition, or the third biological composition, or any combination thereof.
177. A method of analyzing an target object for susceptibility to fibrosis, the method comprising:
   placing the target object, the matrix, and the first biological composition in the first channel of the microfluidic device of any preceding or following embodiment/feature/aspect;
   placing the second biological composition in the second channel of the microfluidic device;
   placing the third biological composition in the third channel of the microfluidic device; and
   analyzing the target object, or the first channel, or both to determine the presence or absence of fibrosis associated with the target object.
178. The method of any preceding or following embodiment/feature/aspect, wherein the analyzing is performed after a predetermined time period following placing the target object, the matrix, the first biological composition, the second biological composition, or the third biological composition, or any combination thereof into the microfluidic device.
179. The method of any preceding or following embodiment/feature/aspect, wherein the predetermined time period is sufficient to allow formation of the gradient of at least one type of molecule.
180. The method of any preceding or following embodiment/feature/aspect, wherein the analyzing is performed two or more times.
181. The method of any preceding or following embodiment/feature/aspect, wherein any two consecutive analyses are separated by a predetermined time period.
182. The method of any preceding or following embodiment/feature/aspect, wherein the analyzing comprises removing one or more components from the microfluidic device.
183. The method of any preceding or following embodiment/feature/aspect, wherein the analyzing comprises an immunoassay.
184. The method of any preceding or following embodiment/feature/aspect, wherein the analyzing comprises use of an antibody.
185. The method of any preceding or following embodiment/feature/aspect, wherein the antibody is labeled.
186. The method of any preceding or following embodiment/feature/aspect, wherein the analyzing comprises application of a primary antibody and a secondary antibody that comprises a detectable label and binds to the primary antibody.
187. The method of any preceding or following embodiment/feature/aspect, wherein the primary antibody binds a collagen, an alpha-smooth muscle actin, or both.
188. The method of any preceding or following embodiment/feature/aspect, wherein the analyzing comprises a histological assay.
189. The method of any preceding or following embodiment/feature/aspect, wherein the analyzing comprises application of picrosirius red dye.
190. The method of any preceding or following embodiment/feature/aspect, wherein the analyzing comprises visually inspecting the target object, or the first channel, or both for one or more indications of fibrosis associated with the target object.
191. The method of any preceding or following embodiment/feature/aspect, wherein the analyzing comprises using microscopy.
192. The method of any preceding or following embodiment/feature/aspect, wherein the analyzing comprises using one or more spectroscopic techniques.
193. The method of any preceding or following embodiment/feature/aspect, wherein the analyzing comprises nonlinear optical (NLO) multi-photon microscopy, second harmonic generation (SHG) imaging, third harmonic generation (THG) imaging, two-photon excited fluorescence (TPEF), two-photon excited fluorescence (TPEF), cell tracker dye imaging, visible light imaging, (ELISA), a Griess assay, histopathology, immunostaining, cleared tissue microscopy, or proteome profiling of homogenized hydrogel, or any combination thereof.
194. The method of any preceding or following embodiment/feature/aspect, wherein the analyzing comprises using the detector to detect one or more indications of fibrosis associated with the target object.
195. The method of any preceding or following embodiment/feature/aspect, wherein the analyzing comprises inserting the microfluidic device into the receptacle.
196. The method of any preceding or following embodiment/feature/aspect further comprising preparing the target object, the matrix, the first biological composition, the second biological composition, or the third biological composition, or any combination thereof from a patient sample.
197. The method of any preceding or following embodiment/feature/aspect further comprising implanting the target object into or onto a patient.
198. The method of any preceding or following embodiment/feature/aspect further comprising implanting a medical device, or a tissue, or both into or onto a patient, wherein the target object is a representative model of the medical device, or the tissue, or both.
199. The method of any preceding or following embodiment/feature/aspect further comprising removing a medical device, or a tissue, or both from a patient, wherein the target object is a representative model of the medical device, or the tissue, or both.
200. The method of any preceding or following embodiment/feature/aspect further comprising administering an anti-fibrosis agent to the patient.
201. The method of any preceding or following embodiment/feature/aspect, wherein the analyzing comprising determining a quantitative measure of fibrosis.
202. The method of any preceding or following embodiment/feature/aspect, wherein the dosage of an anti-fibrosis agent administered to the patient is based on the quantitative measure of fibrosis.
203. The method of any preceding or following embodiment/feature/aspect, wherein the anti-fibrosis agent is administered to the patient before, concurrently with, or after implanting the target object, a tissue associated with the target object, or a medical device associated with the target object, or any combination thereof.
204. The method of any preceding or following embodiment/feature/aspect, further comprising administering one or more therapeutics to the patient in addition to or in the alternative to the anti-fibrosis agent.
205. The method of any preceding or following embodiment/feature/aspect, wherein the patient is a mammalian patient, or a human patient, or both.
206. A method of screening a therapy for efficacy of treating fibrosis, the method comprising:
   placing the target object, the matrix, and the first biological composition in the first channel of the microfluidic device of any preceding or following embodiment/feature/aspect;
   placing the second biological composition in the second channel of the microfluidic device;
   placing the third biological composition in the third channel of the microfluidic device;
   performing the therapy on the microfluidic device;
   analyzing the target object, or the first channel, or both to determine a presence, an absence, or a degree of fibrosis associated with the target object; and
   determining an efficacy of the therapy based on a difference in the presence, the absence, or the degree of fibrosis without performing the therapy.
207. The method of any preceding or following embodiment/feature/aspect, wherein the therapy comprises a therapeutic compound and the performing comprises placing the therapeutic compound in the first channel, the second channel, or the third channel, or any combination thereof.
208. The method of any preceding or following embodiment/feature/aspect, wherein the therapeutic compound comprises a pharmaceutical compound, or a biologic, or both.
209. The method of any preceding or following embodiment/feature/aspect, wherein the therapy comprises a modification of the target object.
210. The method of any preceding or following embodiment/feature/aspect, wherein the therapy comprises placing a medical device in the first channel, the second channel, or the third channel, or any combination thereof.
211. The method of any preceding or following embodiment/feature/aspect, wherein the therapy comprises applying radiation to the microfluidic device.
212. The method of any preceding or following embodiment/feature/aspect, wherein the therapy further comprises treating a patient with the screened therapy.
213. A method of screening a therapy for efficacy of treating cancer, the method comprising:
   placing the target object, the matrix, and the first biological composition in the first channel of the microfluidic device of any preceding or following embodiment/feature/aspect, wherein the target object comprises a neoplastic cell;
   placing the second biological composition in the second channel of the microfluidic device;
   placing the third biological composition in the third channel of the microfluidic device;
   performing the therapy on the microfluidic device;
   analyzing the target object, or the first channel, or both to determine a presence, an absence, or a degree of fibrosis associated with the target object; and
   determining an efficacy of the therapy based on a difference in the presence of, in the absence of, or the degree of, cancer without performing the therapy.
214. A non-transitory computer-readable medium storing a program comprising instructions to perform a method of any preceding or following embodiment/feature/aspect.
215. A non-transitory computer-readable medium storing a program comprising instructions to control the fibrotic diagnostic system of any preceding or following embodiment/feature/aspect, or a subsystem thereof, or a component thereof, or any combination thereof.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. The scope of the disclosure is not limited to the specific values recited when defining a range.

All references cited in this specification are herein incorporated by reference as though each reference was specifically and individually indicated to be incorporated by reference. The citation of any reference is for its disclosure prior to the filing date and should not be construed as an admission that the present disclosure is not entitled to claim any particular subject matter disclosed herein.

It will be understood that each of the elements described above, or two or more together may also find a useful application in other types of methods differing from the type described above. Without further analysis, the foregoing will so fully reveal the gist of the present disclosure that others can readily adapt it for various uses while still falling within the scope of the claimed subject matter. The foregoing embodiments are presented by way of example only.

What is claimed is:

1. A fibrotic diagnostic system comprising:
a microfluidic device comprising:
a device housing defining a device interior;
a plurality of channels within the device interior, the plurality of channels comprising:
a first channel configured to receive a target object, a matrix, and a first biological composition that is configured to promote fibrosis of the target object,
a second channel configured to receive a second biological composition that is configured to promote fibrosis of the target object,
a first septum separating the first and second channels and comprising a first plurality of openings providing fluid communication between the first and second channels,
a third channel configured to receive a third biological composition configured to promote fibrosis of the target object, and
a second septum separating the first and third channels and comprising a second plurality of openings providing fluid communication between the first and third channels; and
a plurality of a channel ports disposed within the device housing that is in fluid communication with the plurality of channels, the plurality of channel ports comprising:
a first set of channel ports in fluid communication with the first channel,
a second set of channel ports in fluid communication with the second channel, and
a third set of channel ports in fluid communication the third channel,
wherein each set of channel ports comprises one or more channel ports.

2. The fibrotic diagnostic system of any preceding claim 1 wherein the system is configured to form a gradient of at least one type of molecule across the first channel, the second channel, or the third channel, or any combination thereof.

3-54. (canceled)

55. The fibrotic diagnostic system of claim 1, wherein the device housing comprises a substrate and the plurality of channels and the plurality of channel ports are formed in the substrate, wherein the substrate comprises a first material and the one or more channel surfaces comprise at least one second material, each of the at least one second material differing from the first material.

56-61. (canceled)

62. The fibrotic diagnostic system of claim 55, wherein the at least one second material is a coating on the first material.

63-71. (canceled)

72. The fibrotic diagnostic system of claim 55, wherein the at least one second material on the first channel comprises a first coating, the at least one second material on the second channel comprises a second coating, and the at least one second material on the third channel comprises a third coating; the first coating, the second, and the third coating differing in a material composition, a thickness, or patterning, or a combination thereof.

73-76. (canceled)

77. The fibrotic diagnostic system of claim 72, wherein the first coating, the second coating, and the second coating comprise a protein, or a nucleic acid, a lipid, or a carbohydrate, a fibronectin, an extracellular matrix-derived composition, a basement membrane extract, a decellularized extracellular matrix, a solubilized basement membrane preparation, a mixture of extracellular matrix proteins, a tumor derived composition, Matrigel, or a hyaluronic acid, or any combination thereof.

78-102. (canceled)

103. The fibrotic diagnostic system of claim 1, wherein the matrix comprises a hydrogel, a fibronectin, an extracellular matrix-derived composition, a basement membrane extract, a decellularized extracellular matrix, a solubilized basement membrane preparation, a mixture of extracellular matrix proteins, a tumor derived composition, Matrigel, or a hyaluronic acid, a natural polymer, an artificial polymer, or a cross-linked polymer, a polysaccharide, a polypeptide, a lipid, a polynucleotide, a hyaluronic acid, a hyaluronic acid methacrylate, a hyaluronic acid diacrylate, a fibronectin, a fibrinogen, a collagen, or a methacrylate collagen, or any combination thereof.

104-106. (canceled)

107. The fibrotic diagnostic system of claim 1, wherein the first biological composition comprises a fibroblast, or an endothelial cell, a blood serum, an ascorbic acid, a retinoic acid, proline, or bleomycin, a biomolecule promoting fibrosis, a complement protein, a blood clotting associated protein, a bacterial cell wall protein, or a lipopolysaccharide (LPS), or any combination thereof.

108-109. (canceled)

110. The fibrotic diagnostic system of claim 1, wherein the second biological composition comprises at least one type of biological cell capable of promoting fibrosis, a factor that promotes differentiation of a biological cell into a type of biological cell capable of promoting fibrosis, a factor that promotes expression of a biomolecule that promotes fibrosis, or a factor that promotes expansion of a type of biological cell capable of promoting fibrosis, blood serum, a platelet, a white blood cell, a macrophage, a monocyte, a peripheral blood mononuclear cell (PBMC), a stem cell, a mast cell, a dendritic cell, a neutrophil, a granulocyte, a T cell, or a B cell, or any combination thereof.

111-112. (canceled)

113. The fibrotic diagnostic system of claim 1, wherein the third biological composition comprises a population of molecules capable of forming a gradient radiating from the third channel into the first channel, or into both the first and second channels.

114. The fibrotic diagnostic system of claim 113, wherein the third biological composition comprises a cytokine, a chemokine, a cell nutrient, a transcription factor, or a growth factor, CCL-2/MCP-1, CCL3, CCL11,fibroblast growth factor (FGF), an interferon, IFN-gamma, an interleukin (IL), IL-4, IL-1α, IL-1β, IL-2, IL-5, IL-6, IL-7, IL-9, IL-13, IL-17A, IL-17F, IL-21, IL-22, IL-25, platelet derived growth factor (PDGF), TGF-alpha, TGF-beta, granulocyte colony-stimulating factor (G-CSF), macrophage colony-stimulating factor (M-CSF), granulocyte macrophage colony-stimulating factor (GM-CSF), prothrombin, or thrombin, or any combination thereof.

115. (canceled)

116. The fibrotic diagnostic system of claim 2, wherein the at least one type of molecule of the gradient comprises a cytokine, a chemokine, a cell nutrient, a transcription factor, or a growth factor, or any combination thereof.

117-124. (canceled)

125. The fibrotic diagnostic system of claim 1, further comprising a detector configured to receive the microfluidic device, the detector comprising a sensor configured to detect fibrosis or characteristic indicative of the same of the target object.

126-128. (canceled)

129. The fibrotic diagnostic system of claim 125, wherein the detector further comprises a thermal subsystem configured to regulate a temperature of the microfluidic device, the thermal subsystem comprising a heater, or a cooler, or both.

130-133. (canceled)

134. The fibrotic diagnostic system of claim 125, wherein the detector further comprising a fluidic subsystem configured for fluidic communication with the microfluidic device, wherein the fluidic subsystem is configured to load the target object, the matrix, the first biological composition, the second biological composition, or the third biological composition, or any combination thereof into the microfluidic device, wherein the fluidic subsystem is configured to supply one or more nutrients to the microfluidic device, wherein the fluidic subsystem is configured to regulate a pressure in an interior of the microfluidic device; wherein the fluidic subsystem is configured to regulate a gradient across the first channel, the second channel, or the third channel, or any combination thereof.

135-143. (canceled)

144. The fibrotic diagnostic system of claim 125, wherein the sensor is configured to detect a particle, or a wave, or both emitted from the target object, or the microfluidic device, or both indicative of fibrosis.

145-147. (canceled)

148. The fibrotic diagnostic system of claim 125, wherein the detector further comprises a particle source, or a wave source, or both configured to transmit a particle, a wave, or both to the target object, or the microfluidic device, or both.

149-160. (canceled)

161. The fibrotic diagnostic system of claim 125, wherein the detector further comprises a processor configured to control the sensor, the source of electromagnetic radiation, the optical subsystem, the thermal subsystem, or the fluidic subsystem, or any combination thereof, wherein the processor is configured to receive the output the signal from the sensor, to receive an operational signal from a user input, or both.

162-176. (canceled)

177. A method of analyzing an target object for susceptibility to fibrosis, the method comprising:
placing the target object, the matrix, and the first biological composition in the first channel of the microfluidic device of claim 1;
placing the second biological composition in the second channel of the microfluidic device;
placing the third biological composition in the third channel of the microfluidic device; and
analyzing the target object, or the first channel, or both to determine the presence or absence of fibrosis associated with the target object.

178-205. (canceled)

206. A method of screening a therapy for efficacy of treating fibrosis, the method comprising:
placing the target object, the matrix, and the first biological composition in the first channel of the microfluidic device of claim 1;
placing the second biological composition in the second channel of the microfluidic device;
placing the third biological composition in the third channel of the microfluidic device;
performing the therapy on the microfluidic device;
analyzing the target object, or the first channel, or both to determine a presence, an absence, or a degree of fibrosis associated with the target object; and
determining an efficacy of the therapy based on a difference in the presence, the absence, or the degree of fibrosis without performing the therapy.

207-215. (canceled)
